(19) United States
(12) Patent Application Publication
(10) Pub. No.: US 2013/0209584 A1
(43) Pub. Date: Aug. 15, 2013

(54) PHARMACEUTICAL FORMULATIONS OF NITRITE AND USES THEREOF

(71) Applicant: TheraVasc Inc., (US)

(72) Inventors: Christopher KEVIL, Shreveport, LA (US); Anthony GIORDANO, Chesterland, OH (US)

(73) Assignee: TheraVasc Inc., Cleveland, OH (US)

(21) Appl. No.: 13/772,127

(22) Filed: Feb. 20, 2013

Related U.S. Application Data
(63) Continuation-in-part of application No. 12/904,791, filed on Oct. 14, 2010.
(60) Provisional application No. 61/251,483, filed on Oct. 14, 2009.

(51) Int. Cl. A61K 33/00

(52) U.S. Cl. CPC A61K 33/00
USPC 424/718

(57) ABSTRACT

The present invention relates to pharmaceutical compositions of nitrites such as inorganic nitrites, or any pharmaceutically acceptable salts, solvates, or prodrugs thereof, and the medical use of these compositions. The pharmaceutical compositions, which can be formulated for oral administration, can provide immediate release or extended release of the nitrite ion (NO₂⁻). The pharmaceutical compositions of the invention are useful, for example, for the treatment of chronic tissue ischemia, in particular peripheral artery disease (PAD).

### CROSS-REFERENCE TO RELATED APPLICATIONS

This application is a continuation-in-part of U.S. patent application Ser. No. 12/904,791, filed on Oct. 14, 2010, which claims benefit of U.S. Provisional Application No. 61/251,483, filed Oct. 14, 2009, which are hereby incorporated by reference.

### BACKGROUND OF THE INVENTION

The present invention relates to pharmaceutical compositions of nitrites and the medical use of these compositions.

Chronic tissue ischemia, i.e., persistent restriction of blood supply to a tissue, can impair tissue function and result in tissue and organ damage, thus contributing significantly to human morbidity and mortality. The chronic tissue ischemia can stem from any of a wide range of medical conditions that result in the persistent or recurring restriction of blood supply to the tissue, e.g., disorders such as peripheral artery disease, type 1 or type 2 diabetes, atherosclerotic cardiovascular disease, intermittent claudication, critical limb ischemic disease, stroke, myocardial infarction, inflammatory bowel disease, and peripheral neuropathy; traumatic injuries such as wounds, burns, lacerations, contusions, bone fractures, infections, or surgical procedures; congenital malformations such as hernias, cardiac defects and gastrointestinal defects. Thus, chronic tissue ischemia can occur in a variety of tissue types including, for example, skeletal muscle, smooth muscle, cardiac muscle, neuronal tissue, skin, mesenchymal tissue, connective tissue, gastrointestinal tissue and bone. Accordingly, there is a continuing need for therapeutic strategies that restore blood supply to affected regions.

### SUMMARY OF THE INVENTION

In general, in a first aspect, the invention features a pharmaceutical composition that includes an effective amount of inorganic nitrite, or a pharmaceutically acceptable salt, solvate, or prodrug thereof, and a pharmaceutically acceptable excipient. Desirably, administration of the pharmaceutical composition to a human results in a plasma concentration of nitrite ion that is maintained between 0.05 μM and 10 μM (e.g., between 0.1 μM and 10 μM, 0.5 μM and 5 μM, 0.1 μM and 3 μM, or 0.1 μM and 1 μM) for up to 14 hours.

In other embodiments, the inorganic nitrite is administered at a dose that is between 0.1 μg-10 mg/kg weight of the human (e.g., between 1 μg-5 mg/kg, 0.05-10 mg/kg, 0.1-5 mg/kg, 0.5-5 mg/kg, 0.5-3 mg/kg, 0.1-1.5 mg/kg, 0.1-0.35 mg/kg, 0.35-0.75 mg/kg, or 0.75-1 mg/kg). In still other embodiments, the dose is 0.25 mg/kg, 0.5 mg/kg, or 1 mg/kg.

In certain embodiments, the pharmaceutical composition includes 0.5-5.0 mmol (e.g., 1.0-4.0 mmol) of nitrite ion (NO₂⁻).

In other embodiments, the nitrite ion is provided as NaNO₂, KNO₂, or arginine nitrite. In certain embodiments, the nitrite ion is provided as NaNO₂.

In still other embodiments, the pharmaceutical composition is formulated for oral administration. In further embodiments, pharmaceutical composition is a tablet or capsule.

In other embodiments, the pharmaceutical composition includes an excipient that is an alkanizing agent, a glidant, a lubricant, a bulking agent, a polymer that comprises cellulose, or polyethylene glycol, or any combination thereof. In still other embodiments, the pharmaceutical composition includes a pharmaceutically acceptable excipient (e.g., a pH sensitive polymer or a biodegradable polymer) for delayed release of the inorganic nitrite, such that, when orally administered to a human subject, the inorganic nitrite is not substantially released in the stomach of the subject. In further embodiments, an enteric coating includes the pharmaceutically acceptable excipient for delayed release of the inorganic nitrite. In certain embodiments, the pharmaceutically acceptable excipient is ethyl cellulose, cellulose acetate, cellulose acetate butyrate, cellulose triacetate, cellulose acetate phthalate (CAP), cellulose trimellitate, hydroxypropylmethylcellulose acetate succinate, or Eudragit® L or S. In further embodiments, the pharmaceutical composition further includes polyethylene glycol and/or a plasticizer.

In some embodiments, the pharmaceutical composition is a multiparticulate dosage form. In certain embodiments, the multiparticulate dosage form includes pellets or granules. In further embodiments, the pellets or granules are coated with a coating layer that includes a biodegradable polymer (e.g., a polysaccharide such as alginate, pectin, carrageenan, chitosan, dextran, shellac, or xanthan gum, or any mixture thereof).

In a second aspect, the invention relates to a pharmaceutical composition formulated for oral administration that includes an effective amount of inorganic nitrite, or a pharmaceutically acceptable salt, solvate, or prodrug thereof, and a pharmaceutically acceptable excipient for delayed release of the inorganic nitrite, such that, when orally administered to a human subject, the inorganic nitrite is not substantially released in the stomach of the subject. In certain embodiments, the pharmaceutical composition is a tablet or capsule.

In a third aspect, the invention features a pharmaceutical composition suitable for oral administration comprising: (a) an effective amount of inorganic nitrite, or a pharmaceutically acceptable salt, solvate, or prodrug thereof; and (b) an enteric coating layer. Desirably, the pharmaceutical composition is formulated such that, when administered to a human subject, the inorganic nitrite is not substantially released in the stomach of the subject.

In certain embodiments, administration of the pharmaceutical composition to a human results in a plasma concentration that is maintained between 0.05 μM and 10 μM (e.g., between 0.1 μM and 10 μM, 0.5 μM and 5 μM, 0.1 μM and 3 μM, or 0.1 μM and 1 μM).

In other embodiments, the inorganic nitrite is administered at a dose that is between 0.1 μg-10 mg/kg weight of the human (e.g., between 1 μg-5 mg/kg, 0.05-10 mg/kg, 0.1-5 mg/kg, 0.5-5 mg/kg, 0.5-3 mg/kg, 0.1-1.5 mg/kg, 0.1-0.35 mg/kg, 0.35-0.75 mg/kg, or 0.75-1 mg/kg). In further embodiments, the dose is 0.25 mg/kg, 0.5 mg/kg, or 1 mg/kg.

In still other embodiments, the pharmaceutical composition includes 0.5-5.0 mmol (e.g., 1.0-4.0 mmol) of nitrite ion (NO₂).

In certain embodiments, the nitrite ion is provided as NaNO₂, KNO₂, or arginine nitrite. In further embodiments, the nitrite ion is provided as NaNO₂.

In other embodiments, the enteric coating layer includes a pharmaceutically acceptable excipient is a pH sensitive polymer or a biodegradable polymer.

In still other embodiments, the pharmaceutical composition is a tablet or capsule.

In any of the foregoing aspects, upon release, the plasma concentration of nitrite ion is maintained for a period of up to 14 hours (e.g., 2-14 hours, 4-14 hours, 6-12 hours, or 6-10 hours). The periods of maintained plasma concentration can occur, e.g., during and/or after the time of peak plasma concentration. In some embodiments, 30-50% of the nitrite ion is released in the first hour and the remainder of the nitrate ion is released in the following 2-14 hours.

In another aspect, the invention features a method for treating or preventing chronic tissue ischemia in a human. Desirably, the method includes the administration of any of the pharmaceutical compositions described herein to a human. In certain embodiments, the administration is oral.

In still another aspect, the invention features a method of supplementing deficits in circulating nitrite found in a patient, wherein said method comprises the administration of any of the pharmaceutical compositions described herein to a human

The present invention relates to pharmaceutical compositions of nitrite (e.g., inorganic nitrite) and use of these compositions for the treatment of chronic tissue ischemia, including chronic tissue ischemia associated with a disorder, trauma or a congenital defect.

As used herein, the term “delayed release” refers to a pharmaceutical preparation, e.g., an orally administered formulation, which passes through the stomach substantially intact and dissolves in the small and/or large intestine (e.g., the colon). In some embodiments, delayed release of the active agent (e.g., nitrite as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably refer to a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” refer to pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “pharmaceutical composition,” as used herein, represents a composition containing a compound described herein (e.g., inorganic nitrite, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, represents those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, means a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, refers to prophylactic treatment or treatment that prevents one or more symptoms or conditions of a disease, disorder, or conditions described herein (e.g., chronic tissue ischemia). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, represents compounds which are rapidly transformed in vivo to the parent compound of the above formula. Prodrugs also encompass bioequivalent compounds that, when administered to a human, lead to the in vivo formation of nitrite ion (NO₂⁻) or nitrous oxide (NO). A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable such as those described in EP 1336602A1, which is herein incorporated by reference.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also refer to delaying the onset of, retarding or reversing the progress of, or alleviating either the disease or condition to which the term applies, or one or more symptoms of such disease or condition.

The term “unit dosage forms” refers to physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” refers to the amount of nitrite ion present in the plasma of a treated subject (e.g., as measured in a rabbit using an assay described below or in a human).

Other features and advantages of the invention will be apparent from the following Detailed Description, the drawings, and the claims.

### BRIEF DESCRIPTION OF THE DRAWINGS

The application file contains drawings executed in colors (FIGS. 14A, 14B, 15, 16A, 16B, 17A, 17B, and 18). Copies of this patent or patent application with color drawings will be provided by the Office upon request and payment of the necessary fee.

FIGS. 1-10 show the results from simulations of nitrite plasma levels from controlled release formulation 1 (FIG. 1), formulation 2 (FIG. 2), formulation 5 (FIG. 3), formulation 9 (FIG. 4), formulation 9C (FIG. 5), formulation 10C (FIG. 6), formulation 12 (FIG. 7), formulation 12C (FIG. 8), and formulation 13 (FIG. 9), as well as from a control immediate release formulation (FIG. 10). Note in FIG. 9: This polynomial fit predicts only 27% of the 80 mg tablet contents is released over an 8 hour period (21.6 mg released). The simulation assumes that only 27% of the 80 mg dose is released within 8 hours, and then the tablet releases no more material. The maximum possible release predicted by this polynomial is approximately 29%, which requires approximately 11.5 hours.

FIG. 11 shows the release profile of total NOx for formulations 100A, 200A, and 300A in rabbits.

FIG. 12 shows the release profile of nitrate, nitrosothiols, nitrosoheme, and nitrosamines for formulations 100A, 200A, and 300A in rabbits.

FIG. 13 shows the release profile of free nitrite for formulations 100A, 200A, and 300A in rabbits.

FIGS. 14A-14B show data for flow mediated dilation (FMD) in the placebo, 40 mg, and 80 mg group. FIG. 14A shows the least square means change in FMD by least square means. FIG. 14B shows the means change in FMD.

FIG. 15 show data for the 6 minute walk in the placebo, 40 mg, and 80 mg group.

FIGS. 16A-16B show the results from the RAND 36 Questionnaire. FIG. 16A shows results from the physical quality of life assessment in the placebo, 40 mg, and 80 mg group. FIG. 16B shows results from the psychological quality of life assessment in the placebo, 40 mg, and 80 mg group.

FIGS. 17A-17B show results from the WIQ. FIG. 17A shows results from the WIQ in the FAS population. FIG. 17Bs show results from the WIQ in the diabetic population.

FIG. 18 is a graph showing the % Methemoglobin at 30 minutes post-dosing for V1-V8.

FIG. 19 is a flow chart showing the dosing arms for treatment of subjects.

### DETAILED DESCRIPTION

The invention features physiologically acceptable compositions of nitrite, such as inorganic nitrite, and methods by which the compositions can be administered to a patient diagnosed as having, for example, a chronic tissue ischemic disorder, in particular peripheral artery disease (PAD).

### Nitrite

Inorganic Nitrite

The pharmaceutically acceptable compositions of the invention include inorganic nitrite, e.g., a salt or ester of nitrous acid (HNO₂), or a pharmaceutically acceptable salt thereof. Nitrite salts can include, without limitation, salts of alkali metals, e.g., sodium, potassium; salts of alkaline earth metals, e.g., calcium, magnesium, and barium; and salts of organic bases, e.g., amine bases and inorganic bases. Compounds of the invention also include all isotopes of atoms occurring in the intermediate or final compounds. Isotopes include those atoms having the same atomic number but different mass numbers. For example, isotopes of hydrogen include tritium and deuterium. The term “compound,” as used herein with respect to any inorganic nitrite or pharmaceutically acceptable salt, solvate, or prodrug thereof. All compounds, and pharmaceutical acceptable salts thereof, are also meant to include solvated (e.g., hydrated) forms. Nitrite has the chemical formula NO₂⁻ and may exist as an ion in water. Sodium nitrite has the chemical formula NaNO₂ and typically dissolves in water to form the sodium ion Na⁺ and the nitrite ion NO₂⁻. It will further be understood that the present invention encompasses all such solvated forms (e.g., hydrates) of the nitrite compounds. Exemplary nitrite compounds are described in WO 2008/105730, which is hereby incorporated by reference.

In addition to sodium nitrite, representative inorganic nitrite compounds include: ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II) nitrite (Co(NO₂)₂), cobalt(III) potassium nitrite (CoK₃(NO₂)₆; e.g., cobalt(III) potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), postassium nitrite (KNO₂), rubidium nitrite (RbNO₂), silver(I) nitrite (AgNO₂), strontium nitrite (Sr(NO₂)₂), and zinc nitrite (Zn(NO₂)₂).

The compounds of the present invention can be prepared in a variety of ways known to one of ordinary skill in the art of chemical synthesis. Methods for preparing nitrite salts are well known in the art and a wide range of precursors and nitrite salts are readily available commercially. Nitrites of the alkali and alkaline earth metals can be synthesized by reacting a mixture of nitrogen monoxide (NO) and nitrogen dioxide (NO₂) with a corresponding metal hydroxide solution, as well as through the thermal decomposition of the corresponding nitrate. Other nitrites are available through the reduction of the corresponding nitrates.

The present compounds can be prepared from readily available starting materials using the methods and procedures known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one of ordinary skill in the art by routine optimization procedures.

Suitable pharmaceutically acceptable salts include, for example, sodium nitrite, potassium nitrite, or calcium nitrite. Still other exemplary salts are found in Remington's Pharmaceutical Sciences, 17th ed., Mack Publishing Company, Easton, Pa., 1985, p. 1418, Berge et al., J. Pharmaceutical Sciences 66:1-19, 1977 and Pharmaceutical Salts Properties, Selection, and Use, (Eds. P. H. Stahl and C. G. Wermuth), Wiley-VCH, 2008, each of which is incorporated herein by reference in its entirety.

### Pharmaceutical Compositions

The pharmaceutically acceptable compositions of the invention include inorganic nitrite, e.g., a salt of nitrous acid (HNO₂) such as NaNO₂, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

The pharmaceutical composition can include nitrate salts, or prodrugs thereof, or other therapeutic agents. Exemplary nitrate salts are described in WO 2008/105730. Exemplary therapeutic agents that may be included in the compositions described herein are cardiovascular therapeutics (e.g., anti-thrombotics (e.g. dipyridamole, clopidogrel, and the like), anti-hypertensives (e.g., Ca⁺⁺ channel blockers, AT-2 blockers, ACE inhibitors, and the like), anti-cholesterols (e.g., statins, fibrates, and the like), and thiazolidinedione therapeutics.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Formulations for Colonic Drug Release

In some embodiments, colon-targeted drug delivery systems can be used. Exemplary approaches include, but are not limited to:
(a) covalent linkage of the drug with the carrier to form a prodrug that is stable in the stomach and small intestine and releases the drug in the large intestine upon enzymatic transformation by the intestinal microflora; examples of these prodrugs include azo-conjugates, cyclodextrin-conjugates, glycoside-conjugates, glucuronate conjugates, dextran-conjugates, polypeptide and polymeric conjugates;
(b) approaches to deliver intact molecule to the colon, such as coating with pH-sensitive polymers to release the drug at neutral to alkaline pH, or coating with biodegradable polymers which release the drug upon degradation by the bacteria in the colon;
(c) embedding the drug in biodegradable matrices and hydrogels which release the drug in response to the pH or biodegradation;
(d) time released systems where once the multicoated formulation passes the stomach, the drug is released after a lag time of 3-5 hrs which is equivalent to the transit time of the small intestine;
(e) using redox-sensitive polymers where a combination of azo and disulfide polymers, provide drug release in response to the redox potential of the colon;
(f) using bioadhesive polymers which selectively adhere to the colonic mucosa slowly releasing the drug; and
(g) osmotic controlled drug delivery where the drug is released through semi-permeable membrane due to osmotic pressure.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, i.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Dosing Regimes

The present methods for treating chronic tissue ischemia are carried out by administering an inorganic nitrite for a time and in an amount sufficient to result in the growth of new blood vessels in the ischemic tissue.

The amount and frequency of administration of the compositions can vary depending on, for example, what is being administered, the state of the patient, and the manner of administration. In therapeutic applications, compositions can be administered to a patient suffering from chronic tissue ischemia in an amount sufficient to relieve or least partially relieve the symptoms of chronic tissue ischemia and its complications. The dosage is likely to depend on such variables as the type and extent of progression of the chronic tissue ischemia, the severity of the chronic tissue ischemia, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of chronic tissue ischemia or slowing its progression.

The amount of inorganic nitrite per dose can vary. For example, a subject can receive from about 0.1 μg/kg to about 10,000 μg/kg. Generally, the nitrite is administered in an amount such that the peak plasma concentration ranges from 150 nM-250 μM. Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, or 1° mg/kg. Exemplary peak plasma concentrations can range from 0.05-10 μM, 0.1-10 μM, 0.1-5.0 μM, or 0.1-1 μM. The peak plasma concentrations may be maintained for 6-14 hours, e.g., for 6-12 or 6-10 hours.

The frequency of treatment may also vary. The subject can be treated one or more times per day (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce chronic tissue ischemia.

### Methods of Treatment

### Nitrite as Nutritional Supplementation

Plasma nitrite levels have been shown to be inversely correlated to cardiovascular risk factors, with subjects having the greatest number of risk factors, having the lowest level of plasma nitrites (Kleinbongard et al., Free Radical Biology & Medicine 40:295-302, 2006). In normal subjects, exercise results in a release of stored nitrite to the plasma, increasing plasma nitrite levels; however, in diabetic and PAD patients, exercise does not increase the level of plasma nitrite and in fact, leads to a further decrease in circulating nitrite levels (Allen et al., Nitric Oxide 20:231-237, 2009). Thus, a nutritional supplementation of nitrite might be effective in overcoming these deficits in plasma nitrite levels in cardiovascular and vascular disorders and given the relationship of nitrite to nitric oxide, the deficits in nitric oxide found in these diseases or due to dietary deficiencies in nitrite.

The present invention provides nutritional compositions of nitrite, e.g., inorganic nitrite, or a pharmaceutically acceptable prodrug thereof, for both prophylactic and therapeutic nutritional supplementation, specifically in cardiovascular, metabolic, inflammatory or vascular diseases. Specifically, the present invention relates to novel compositions of nitrite, e.g., inorganic nitrite, or a pharmaceutically acceptable prodrug thereof, that can be used to supplement the nutritional deficiencies observed in patients with diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy, stem cell diseases, and/or dietary restrictions. In addition, the compositions may be used to treat the nutritional deficiencies of patients suffering from a disease state that results in decreased plasma nitrite or nitric oxide levels.

### Inflammatory Diseases

The pharmaceutical compositions and methods described herein can be used to treat innate and acquired inflammatory diseases. The inflammatory diseases encompassed by the methods of this invention can stem from a wide range of medical conditions that cause inflammation. One type of inflammatory diseases which can be treated by the compositions and methods described in this invention are immuno-inflammatory diseases. Examples of immuno-inflammatory diseases include rheumatoid arthritis, juvenile rheumatoid arthritis, osteoarthritis, transplant rejection, sepsis, acute respiratory distress syndrome, asthma, and cancer. Another type of inflammatory diseases which can be treated by the composition and methods described in this invention are the autoimmune diseases. Examples of autoimmune diseases include such conditions as multiple sclerosis, psoriasis, inflammatory bowel disease, glomerulonephritis, lupus, uveitis, and chronic hepatitis. Other inflammatory diseases can also be treated by the compositions and methods described in this invention, including such conditions caused by trauma, oxidative stress, cell death, irradiation damage, ischemia, reperfusion, cancer, transplant rejection, and viral infection.

### Tissue Regeneration

The pharmaceutical compositions and methods described herein can be used to stimulate tissue regeneration, e.g., following damage to a tissue or organ caused by such conditions as trauma, scarring, abnormal protein deposition, amyloidoses, ischemia or diabetes, infections, or surgical procedures; congenital malformations such as hernias, cardiac defects and gastrointestinal defects that result in damage to the tissue.

### Chronic Tissue Ischemia

Chronic tissue ischemia is associated with a wide range of medical conditions that result in partial, substantially complete or complete reduction of blood flow to a body part or tissue comprising a body part and may be the result of disease, injury, or of an unknown cause, and may be influenced by one's genetic constitution. Regardless of the medical condition leading to chronic tissue ischemia, a patient who has chronic tissue ischemia is a candidate for treatment with the pharmaceutically acceptable compositions comprising inorganic nitrite described herein. Treatment can completely or partially abolish some or all of the signs and symptoms of chronic tissue ischemia, decrease the severity of the symptoms, delay their onset, or lessen the progression or severity of subsequently developed symptoms.

New Blood Vessel Growth

As described further below, the compositions of the invention are administered for a time and in an amount sufficient to result in the growth of new blood vessels in the ischemic tissue. We may use the terms “new blood vessel growth,” “new blood vessel formation” and “new blood vessel development” interchangeably. New blood vessel growth refers all phases of the process of blood vessel formation, including the initial signaling events, cellular recruitment of endothelial cells, the formation and enlargement of new vessels and connection of new vessels with pre-existing vessels. The new blood vessel growth may stem from any process that results in revascularization or neovascularization of the ischemic tissue, for example, angiogenesis, or arteriogenesis, or a combination of angiogenesis and arteriogenesis. The term vasculogenesis typically is used to describe the embryonic development of blood vessels from angioblasts. Angiogenesis is generally understood to be a post-natal physiologic process required for would healing. Angiogenesis generally encompasses the formation of new capillaries or capillary branches by sprouting, budding and intussusception from pre-existent capillaries. Arteriogenesis i.e., the growth of preexisting arteriolar connections into true collateral arteries, is generally understood to encompass the formation of mature arteries from pre-existent interconnecting arterioles after an arterial occlusion. It shares some features with angiogenesis, but the pathways leading to it can differ, as do the final results: arteriogenesis is potentially able to fully replace an occluded artery whereas angiogenesis typically cannot. Increasing the number of capillaries within the ischemic region cannot increase blood flow when the limiting structure lies upstream of the new capillaries; formation of new collateral vessels that divert blood flow around the site of a blockage. In addition, the structures produced by angiogenesis and arteriogenesis differ in their cellular composition. Capillaries are tubes formed by endothelial cells which are supported by vascular pericytes. Arteries and veins are tubes that consist of multiple layers: the intima, which is composed of endothelial cells, pericytes, and a basement membrane; the media, which is composed principally of smooth muscle cells and their extracellular matrix; and, in the largest vessels, the adventitia, which is composed principally of fibroblasts and their extracellular matrix.

### Chronic Tissue Ischemia

Methods of the invention are applicable to any of a wide range of medical conditions which have as their underlying feature a persistent reduction of or partial or complete blockage of blood flow to a tissue or organ. Thus, the methods are applicable to treatment of chronic tissue ischemia associated with a disorder, with a trauma or an environmental stress. The reduction in blood flow to a tissue can be, for example, the result of a progressive blockage of an artery due to hardening and/or loss of elasticity due to an atheromatous plaque or the presence of a clot. Reduction of blood flow to a tissue can also be the result of an environmental insult, for example, a traumatic injury or surgical procedure that interrupts the blood flow to a tissue or organ. Typically, the oxygen tension of a wound quickly and progressively decreases with the development of varying degrees of hypoxia throughout the wound region. Environmental conditions that induce hypoxia are also within the scope of the invention.

Disorders encompassed by the invention include, for example, cardiovascular disease, peripheral artery disease, arteriosclerosis, atherosclerotic cardiovascular disease, myocardial infarction, critical limb ischemic disease, stroke, acute coronary syndrome, intermittent claudication, diabetes, including type 1 and type 2 diabetes, skin ulcers, peripheral neuropathy, inflammatory bowel disease, ulcerative colitis, Crohn's disease, intestinal ischemia, and chronic mesenteric ischemia. The methods of the invention are also applicable to chronic tissue ischemia associated with a trauma, for example, a traumatic injury such as a wound, laceration, burn, contusion, bone fracture or chronic infection. Also encompassed by the invention are tissue injuries sustained as part of any surgical procedure, for example, endarterectomy. Procedures involving tissue or organ transplantation are within the scope of the invention. Examples include vascular bypass grafts, heart, liver, lung, pancreatic islet cell transplantation as well as transplantation of tissues generated ex vivo for implantation in a host. The methods of the invention are also useful for treating a chronic ischemic condition brought about by exposure to an environmental insult, for example, chronic exposure to hypoxic conditions e.g., high altitude, or sustained aerobic exertion.

The methods provided herein are applicable to any of a wide range of tissue types including, for example, muscle, smooth muscle, skeletal muscle, cardiac muscle, neuronal tissue, skin, mesechymal tissue, connective tissue, gastrointestinal tissue or bone. Soft tissue, such as epithelial tissue, e.g., simple squamous epithelia, stratified squamous epithelia, cuboidal epithelia, or columnar epithelia, loose connective tissue (also known as areolar connective tissue), fibrous connective tissue, such as tendons, which attach muscles to bone, and ligaments, which join bones together at the joints.

Thus, for example symptoms of chronic tissue ischemia in peripheral artery disease (PAD), a form of peripheral vascular disease in which there is partial or total blockage of an artery, usually due to atherosclerosis in a vessel or vessels leading to a leg or arm, can include intermittent claudication, that is, fatigue, cramping, and pain in the hip, buttock, thigh, knee, shin, or upper foot during exertion that goes away with rest, claudication during rest, numbness, tingling, or coldness in the lower legs or feet, neuropathy, or defective tissue wound healing. PAD in the lower limb is often associated with diabetes, particularly type 2 diabetes. Arm artery disease is usually not due to atherosclerosis but to other conditions such as an autoimmune disease, a blood clot, radiation therapy, Raynaud's disease, repetitive motion, and trauma. Common symptoms when the arm is in motion include discomfort, heaviness, tiredness, cramping and finger pain. PAD can be diagnosed by performing one or more diagnostic tests including, for example, an ankle brachial index (ABI) test, angiography, ultrasound, or MRI analysis.

Myocardial ischemia can have few or no symptoms, although typically, it is associated with symptoms such as angina, pain, fatigue elevated blood pressure. Diagnostic tests for myocardial ischemia include: angiography, resting, exercise, or ambulatory electrocardiograms; scintigraphic studies (radioactive heart scans); echocardiography; coronary angiography; and, rarely, positron emission tomography.

Peripheral artery disease (PAD)

The pharmaceutical compositions and methods described herein are useful in treating peripheral artery disease (PAD). PAD is a manifestation of systemic atherosclerosis and a strong predictor of cardiovascular (CV) mortality. The systemic disease of atherosclerosis in these patients results in arterial stenoses in the arteries supplying the muscles of the lower extremities. During exercise, the stenoses limit the ability to increase blood flow, which leads to an oxygen supply/metabolic demand mismatch, a bio-energetic deficit, and subsequent muscle contractile dysfunction. Thus, the primary pathophysiology of PAD is related to the limitation in blood flow and abnormal hemodynamics (reduced tissue perfusion pressure and blood flow) of the lower limbs during exercise. Patients with PAD commonly present with symptoms of intermittent claudication (IC), often described by patients as a cramping, aching, or fatigue sensation in the calf muscles of the legs that occurs during physical activity. Notably, the symptom of claudication pain is due to exercise-induced ischemia in the muscles of the leg, causing a significant limitation of functional exercise capacity and adversely affecting quality of life.

The risk of atherosclerotic disease and PAD is markedly increased among individuals with diabetes, and epidemiological data have demonstrated a strong association between diabetes and an increased prevalence of PAD. Insulin resistance and the metabolic sequelae of diabetes are considered major contributors to the high prevalence of cardiovascular diseases (CVD) and CV events in this population. Of particular concern is that in the presence of diabetes and PAD, these patients are at increased risk for disease progression to critical leg ischemia (CLI), lower extremity amputation, and cardiovascular events than their non-diabetic counterparts.

Dysfunction of the endothelium is an early event in the development of atherosclerosis and is associated with the presence of cardiovascular risk factors, diabetes, and cardiovascular diseases, including PAD. A hallmark feature of endothelial dysfunction in these conditions is abnormal vascular reactivity, mediated, in part, by reduced levels of endothelium-dependent nitric oxide (NO). Under basal conditions, NO is produced in vivo by both enzymatic and non-enzymatic processes.

Enzymatic NO formation occurs via the interaction of L-arginine and one of 3 isoforms of nitric oxide synthase (NOS) (e.g., endothelial (eNOS), neuronal (nNOS) or inducible (iNOS). Endothelial-derived NO plays an essential role in regulating normal vascular function by stimulating NO-dependent activation of soluble guanylate cyclase (sGC) leading to the activation of a signaling cascade causing smooth muscle relaxation and vasodilation. Nitric oxide also acts as an important signaling molecule mediating vascular inflammation, angiogenesis, and cellular respiration. A consistent feature in vascular diseases, as well as diabetes, is dysfunction in NO-dependent signaling processes, occurring either through a deficit in NO synthesis, NO bioavailability, or both. Clinically, endothelial dysfunction can be assessed non-invasively using ultrasound techniques of flow-mediated vasodilation of the conduit arteries, and studies have demonstrated endothelial-derived NO production to be reduced in diabetes and PAD. Thus, compromised NO-bioactivity has been advanced as a significant contributor to the abnormal physiological responses and poor clinical outcomes in these diseased populations.

Recently, interest has focused on non-enzymatic sources of NO which may be amenable to therapeutic manipulation. For example, the metabolic products of NO metabolism such as nitrite and nitrate, once thought of as NO metabolism end-products, may serve as an alternative source of NO that can be readily converted to active NO under certain physiological conditions, such as hypoxia and ischemia. Nitrite is a first order metabolite of NO oxidation and a marker of constitutive NOS activity. More recently, nitrite has been advanced as a circulating NO storage depot and delivery source, reacting with oxyhemoglobin to form nitrate and methemoglobin (met-Hb) or with deoxyhemoglobin to form NO, nitrosylhemoglobin, and other NO adducts. Since nitrite is found ubiquitously in the systemic circulation, the dual fates of nitrite metabolism position it as a unique physiological source of NO that may target pathophysiological conditions, such as tissue ischemia. Indeed, circulating plasma nitrite levels are found to be reduced in patients with diabetes and PAD.

Recent data describe a net loss of nitrite stores following exercise in both diabetic PAD and PAD-only patients compared with healthy individuals. These results suggest a significant decrease in the NO pool during periods of exercise-induced ischemia in affected patients, consistent with a depletion of NO stores in an attempt to normalize blood flow and oxygen delivery. Over time and the intermittent periods of ischemia in these patients, it is conceivable that NO stores may become depleted, contributing to the abnormal circulatory responses and systemic endothelial function of this patient group.

Restoring or repleting NO bioavailability may therefore represent a critical therapeutic goal. Although many structurally and chemically diverse NO-donor compounds have been synthesized and used widely in experimental studies, no NO-donor has been approved for use in the clinic. This limitation stems largely from the inability of NO-donor compounds to deliver NO to specific sites, the consequences being that NO-donors elicit systemic vascular effects resulting in hypotension. Because of the selective nature of nitrite's metabolism yet ubiquitous nature, supplemental nitrite has been postulated as a uniquely positioned NO-donor and therapeutic approach in the treatment of cardiovascular conditions.

10

Combination Therapy/Treatment The method of the invention can also be used in conjunction with other remedies known in the art that are used to treat chronic tissue ischemia including, drug therapy, surgery, anti-inflammatory agents, antibodies, exercise, or lifestyle changes. The choice of specific treatment may vary and will depend upon the severity of the chronic tissue ischemia, the subject's general health and the judgment of the attending clinician.

The present compositions can also be formulated in combination with one or more additional active ingredients, which can include any pharmaceutical agent such antihypertensives, anti-diabetic agents, statins, anti-platelet agents (clopidogrel and cilostazol), antibodies, immune suppressants, anti-inflammatory agents, antibiotics, chemotherapeutics, and the like. In some embodiments, the composition also includes an inorganic nitrate; in other embodiments, the composition excludes inorganic nitrates. For example, the present composition can include inorganic nitrite and nitrates in a ratio that is between 1-5 to 1-100 nitrite:nitrate, e.g., 1-5,1-10, 1-30, 1-50, 1-70, or 1-100 nitrite:nitrate.

### EXAMPLES

Controlled Release Pharmaceutical Formulations

Exemplary formulations for oral administration include tablet and capsule formulations. For example, the powdered components described for a tablet formulation can be used to prepare a capsule formulation, a suitable capsule size depending on the dose of the active and density of the fill, such as size 1, 0, or 00 capsules. In some embodiments, the table or capsule may not have an enteric coating. In other embodiments, the pharmaceutical compositions of the invention can be formulated for controlled release of nitrite ion. If a capsule is described as coated, the coating can be applied to the capsule after filling. Capsule formulations can optionally employ self-locking capsule shells (e.g., Coni-Snap®, Posilok®, Snap-Fit®, or the like) for ease of handling during the coating process.

The exemplary compositions include between 0.5-4.0 mmol of total nitrite ion; specifically, between 1.8-3.6 mmol of NaNO₂. The compositions can include any prodrug of nitrite thereof, e.g., 125-250 mg of NaNO₂, 154-308 mg of KNO₂, or 201-402 mg of arginine nitrite. The amount of nitrite ion used in the pharmaceutical compositions can be varied as described herein. For example, the formulations can also include any of the excipients described herein, preferably an alkanizing agent (e.g., sodium bicarbonate or calcium carbonate), a glidant (e.g., fumed silica), a lubricant (a fatty acid salt (e.g., magnesium stearate), a pure solid fatty acid, or solid polyethylene glycol), or a bulking agent with good flow properties (e.g., silicified microcrystalline cellulose (Prosoly® SMCC90)). The compositions can also include any of the excipients described for use in compositions that are formulated for enteric release, e.g., in enteric formulations. Formulations can also include rate-controlling polymer coatings (e.g., ethyl cellulose, cellulose acetate, cellulose acetate butyrate, cellulose triacetate and the like, which can be combined with PEG-4000). If desired, the amount of PEG-4000 used can be varied in order to generate aqueous pores in the coat through which the sodium nitrite can diffuse. Enteric polymer coatings can also be used, and exemplary polymers include cellulose acetate phthalate (CAP), cellulose trimellitate, hydroxypropylmethylcellulose acetate succinate, Eudragit® L or S, or the like Where a polymer coating is used, the formulation can also include a plasticizer (e.g., triethylcitrate, triacetin, acetyl monoglycerides, or the like). The total enteric coat (polymer+plasticizer) can be added in an amount that, for example, results in a 10% weight gain.

The production and testing of several tablet and pellet formulations for the controlled release of nitrate is described below.

### Tablet Preparation Procedures

All solid components, including sodium nitrite, were weighed to produce tablets with the desired weight ratios of components. Enough powder blend was prepared to prepare 4-5 tablets. The powdered components were thoroughly mixed before compressing into tablets. For tablets containing a waxy component (i.e. Castorwax®), sodium nitrite and other components were dispersed in molten wax and the mixture solidified while mixing to maintain a homogeneous blend. After solidifying, the mixture was ground to powder for further mixing, if required. Mixing of all powdered components was accomplished with a mortar and pestle.

The tablets were compressed on a Carver® Press with a ½″ (1.27 cm) punch and die. A force of 5000 lbs was applied for 30 seconds to obtain tablets for release testing.

The tablet dimensions were:
580 mg tablets: 1.27 cm dia.×0.38 cm thickness (½″× 1/7″) or
480 mg tablets: 1.27 cm dia.×0.32 cm thickness (½″×⅛″)

Tablet thicknesses were dependent on the total weight of powdered components and the nature of the excipients employed. Thus, the thicknesses disclosed varied between 10-15%, depending on the mixture being compressed.

The tablets were carefully pushed from the die after compression and stored in a desiccator until dissolution testing. Some tablets were coated with controlled release or enteric coating materials to alter their release profiles.

### Pellet Preparation Procedures

Small pellets containing 5 mg of sodium nitrite were prepared according to the following procedure for animal testing (oral administration to rabbits). All solid components, including sodium nitrite, were weighed to produce pellets with the desired weight ratios of components. Enough powder blend was prepared to prepare 40-50 pellets.

The powdered components were sieved (150-250 microns) and thoroughly mixed by geometric dilution before compressing into pellets. The pellets were compressed with a Parr Model 2811 pellet press with a 3 mm punch-and-die. The pellet press operated with manual compression and did not allow control of the applied force but did produce cohesive pellets for all formulations. The pellets weighed 23-35 mg depending on the formulation employed. One pellet batch was manually coated with an ethylcellulose/triacetin coating (4/1) which was 11-15% of the pellet weight.

The pellet dimensions were: 3 mm dia.×5-7 mm thickness. Pellet thicknesses were dependent on the total weight of powdered components and the nature of the excipients employed. Thus, the thicknesses disclosed varied about 50% depending on the mixture being compressed.

The pellets were carefully pushed from the die after compression and stored in a desiccator until shipment for animal testing. One pellet batch was coated with a controlled release coating to alter its release profile. The coating procedure is described separately below. The Castorwax pellets were compressed twice. The first compression was at ambient temperature; the second compression was in the 3 mm die after heating the die to 50-60° C. in an oven. The second compression induced better flow of the Castorwax around the sodium nitrite and sodium acetate particles.

### Tablet/Pellet Coating Procedure

Sodium nitrite tablets were coated manually by carefully dropping a measured volume of coating solution on to the tablet and carefully spreading it on the surfaces and edge of the tablet. After solvent evaporation, the process was repeated multiple times until an adequate amount of coating was applied. For pellets and some tablet batches, a dip coating process was employed which involved carefully dipping the pellet/tablet into coating solution and letting it air dry while holding it with forceps. The dipping process was repeated until an adequate amount of coating was applied.

The coatings employed were ethylcellulose (EC) with triacetin as a plasticizer and cellulose acetate phthalate (CAP, Cellacefate, NF). Various ratios of EC and triacetin were employed to obtain coats with different brittleness and different permeabilities to water and sodium nitrite. EC/triacetin was applied to tablets or pellets from solutions that contained chloroform, methylene chlorideor 95% ethanol. CAP was employed as an enteric coating material which was applied to tablets from a dioxane solution. Other coating solvents gave CAP coated tablets which did not withstand simulated gastric fluid for two hours without disintegrating.

### Tablet Components

Sodium nitrite, Certified ACS Reagent, crystalline, Fisher Scientific, Lot #080939A
Polyox® Coagulant, Blend #C-289, 5 million MW, N.F. Grade, Union Carbide,
Polyox® WSR 303, 7 million MW, N.F. Grade, Colorcon
Avicel® PH-302, microcrystalline cellulose, FMC Corporation, Lot #Q939C
Ethocel®, ethylcellulose, Standard 100 premium, Colorcon
Castorwax®, NF, hydrogenated castor oil, CASCHEM, Lot #00121431
Methocel K100M, hydroxypropyl methylcellulose, premium CR grade, Colorcon
Klucel® HXAF Pharm., hydroxypropylcellulose, 1.15 million MW, Aqualon Division, Hercules, Inc.
Klucel® MF Pharm., hydroxypropylcellulose, 850,000 MW, Aqualon Division, Hercules, Inc.
Sodium Chloride, Certified ACS Reagent, Fisher Scientific
Sodium Acetate Trihydrate, ACS Reagent, Fisher Scientific

### Release Testing Procedure for Tablets

The USP paddle method was employed at 50 RPM stirring for all nitrite release testing. A Vankel® USP 6-station dissolution apparatus was used. A volume of 500 mL distilled water at 37° C. was used as the release medium in each release vessel. Tablet release studies were conducted in duplicate or triplicate for each formulation tested.

Samples (35 mL) of the release medium were taken from each vessel at regular time intervals (typically ½, 1, 2, 3, 4 hours (or longer). The medium was replenished with 35 mL of distilled water.

At the end of a release run tablets were crushed and allowed to completely release their sodium nitrite content dissolved to determine the total sodium nitrite content in the tablet.

### Sodium Nitrite Release Assay

The UV absorbance at 355 nm was measured with a HewlettPackard® 8453 diode-array UV-visible spectrophotometer for each release sample in a 10-cm quartz cuvette.

From a previously prepared calibration plot, the concentration of sodium nitrite in each sample was calculated and converted to total amount and percent released for each tablet. The average percent released and standard deviation were calculated for two or three tablets run simultaneously. The average percent released vs. time profiles were plotted for each formulation.

The formulations and release profiles of the tablets and pellets produced by the above methods are set forth in tables 1-7.

TABLE 1
Polyox tablet compositions (mg/tablet)

| Fomulation | Polyox | Polyox WSR | Avicel PH | Sodium | Ethylcellulose/Triacetin | Total |
|---|---|---|---|---|---|---|
| No. | Coagulant | 303 | 302 | nitrite | Coating | weight |
| 1 | 200 | 200 | 100 | 80 | — | 580 |
| 9 | 100 | 300 | 100 | 80 | — | 580 |
| 9 C | 100 | 300 | 100 | 80 | 87 (13% w/w) | 667 |
| 14 | 0 | 400 | 0 | 80 | — | 480 |

Formulation 9 C is the same as Formulation 9 except that a 13% coating of ethylcellulose 100/triacetin ( 1/10) was applied to the tablet from a 95% ethanol solution.

TABLE 2
Polyox tablet release results

| Time | | | | | Formulation | | Formulation | |
|---|---|---|---|---|---|---|---|---|
| (hours) | Formulation 1 | % SD | Formulation 9 | % SD | 9 C | % SD | 14 | % SD |
| 1 | 41.9% | 7.0% | 29.1% | 8.3% | 8.2% | 0.4% | 33.6% | 8.3% |
| 2 | 54.1% | 5.1% | 47.9% | 9.0% | 20.7% | 0.8% | 59.7% | 11.0% |
| 3 | 66.2% | 3.2% | 64.2% | 8.3% | 34.0% | 2.9% | 75.5% | 9.7% |
| 4 | 76.9% | 2.5% | 75.2% | 6.3% | 48.4% | 5.9% | 86.4% | 6.7% |
| 6 | 91.0% | 0.7% | 91.7% | 3.7% | 74.6% | 9.4% | 93.5% | 2.4% |
| 8 | 100.0% | 0.0% | 100.0% | 0.0% | 100% | 9.0% | 100.0% | 0.0% |

TABLE 3
Pellet compositions for animal studies (mg/pellet)

| Fomulation | Polyox WSR | | Sodium | | Sodium | Ethylcellulose/Triacetin | Total |
|---|---|---|---|---|---|---|---|
| No. | 303 | Castorwax | Acetate | Hydroxypropylcellulose | nitrite | Coating | weight |
| 100A | 25 | — | | — | 5 | — | 30 |
| 200A | — | 12 | 6 | — | 5 | — | 23 |
| 300A | — | — | — | 25 | 5 | 4-5 (11-15% w/w) | 34-35 |

Formulation 200A was compressed twice. The first compression was at ambient temperature. The second compression was in the 3 mm die after heating the die to 50-60° C. in an oven. Formulation 300A was dip coated with an ethylcellulose 100/triacetin (4/1) coating solution with 95% ethanol as the solvent.

TABLE 4
Castorwax tablet compositions (mg/tablet)

| Fomulation | | HPMC | Sodium | Sodium | Sodium | Ethylcellulose/Triacetin | Total |
|---|---|---|---|---|---|---|---|
| No. | Castorwax | (K100M) | Chloride | Acetate | nitrite | Coating | weight |
| 12 | 200 | 200 | — | — | 80 | — | 480 |
| 12 C | 200 | 200 | | — | 80 | 71 (14.8%) | 551 |
| 13 | 300 | — | 100 | — | 80 | — | 480 |
| 15 | 200 | — | — | 100 | 80 | — | 380 |
| 15 C1 | 200 | — | — | 100 | 80 | 36.5 (9.6%) | 416.5 |
| 15 C2 | 200 | — | — | 100 | 80 | 16 (4.15%) | 396 |

TABLE 5
Castorwax tablet release results

| Time | Formulation | | Formulation | | Formulation | | Formulation | | Formulation | | Formulation | |
|---|---|---|---|---|---|---|---|---|---|---|---|---|
| (hours) | 12 | % SD | 12 C | % SD | 13 | % SD | 15 | % SD | 15 C1 | % SD | 15 C2 | % SD |
| 0.5 | — | — | — | — | — | — | — | — | 0.8% | 0.0% | — | — |
| 1 | 36.0% | 2.9% | 29.1% | — | 17.3% | 1.6% | 58.8% | 0.7% | 2.3% | 0.4% | 2.4% | 0.4% |
| 2 | 53.7% | 3.8% | 46.6% | — | 22.0% | 3.1% | 81.7% | 0.1% | 5.8% | 1.0% | 4.5% | 0.4% |
| 3 | 69.5% | 2.6% | 67.3% | — | 24.3% | 4.0% | 94.8% | 0.8% | 11.5% | 0.3% | 7.9% | 0.6% |
| 4 | 76.6% | 8.6% | 74.1% | — | 27.3% | 3.8% | 100.8% | 0.5% | — | — | 11.8% | 1.4% |
| 6 | 95.8% | 3.9% | 99.5% | — | 32.0% | 4.5% | 100.0% | 0.0% | — | — | 18.2% | 2.2% |
| 8 | 100.0% | 0.0% | 100.0% | — | 35.5% | 4.6% | — | — | 31.9% | 0.7% | 27.5% | 2.9% |
| 24 | — | — | — | — | — | — | — | — | 75.5% | 0.1% | 71.0% | 2.7% |
| 26 | — | — | — | — | 57.1% | 3.0% | — | — | — | — | — | — |

Formulation 12 C is the same as Formulation 12 except that a 14.8% coating of ethylcellulose 100/triacetin ( 1/10) was applied to the tablet from a 95% ethanol solution.
Formulations 13, 15, 15 C1 and 15C2 were prepared by mixing sodium nitrite and other components into melted Castorwax. The molten mass was mixed while solidifying and then ground into a powder with a mortar and pestle before compressing into tablets.
Formulations 15 C1 and 15 C2 are the same as Formulation 15 except that a 9.6% (15 C1) or a 4.15% (15 C2) coating of ethylcellulose 100/triacetin (4/1) was applied to the tablets from a chloroform.

TABLE 6
Ethylcellulose and HPMC tablet compositions (mg/tablet)

| | | | | | | | Ethylcellulose/ | |
|---|---|---|---|---|---|---|---|---|
| Formulation | | HPMC | HPMC | Avicel | HPC | Sodium | Triacetin | Total |
| No. | Ethylcellulose | (K100M) | (K15M) | PH-302 | MF | nitrite | Coating | weight |
| 2 | 400 | — | — | — | | 80 | — | 480 |
| 5 | 200 | 200 | — | — | | 80 | — | 480 |
| 10 C | — | 200 | — | 200 | | 80 | 76 (15.8%) | 556 |
| 16 | — | — | 400 | — | | 80 | — | 480 |
| 17 C | — | — | — | — | 400 | 80 | 41.5 (8.65%) | 521.5 |

TABLE 7
Ethylcellulose and HPMC tablet release results

| Time | | | | | Formulation | | Formulation | | Formulation | |
|---|---|---|---|---|---|---|---|---|---|---|
| (hours) | Formulation 2 | % SD | Formulation 5 | % SD | 10 C | % SD | 16 | % SD | 17 C | % SD |
| 0.5 | — | — | — | — | — | — | — | — | — | — |
| 1 | 56.0% | 1.4% | 50.0% | 2.3% | 24.7% | — | 38.2% | 1.1% | 0.8% | 0.7% |
| 2 | 73.8% | 2.3% | 68.8% | 5.3% | 60.6% | — | 53.0% | 1.3% | 1.1% | 1.0% |
| 3 | 84.3% | 2.1% | 81.2% | 5.1% | 79.2% | — | 78.9% | 8.6% | 2.9% | 1.6% |
| 4 | 91.1% | 2.1% | 89.2% | 4.8% | 87.7% | — | 88.2% | 5.4% | 6.7% | 0.9% |
| 6 | 96.6% | 0.2% | 95.3% | 2.2% | 97.1% | — | 96.3% | 4.1% | 18.0% | 0.5% |
| 8 | 100.0% | 0.0% | 100.0% | 0.0% | 100.0% | — | — | — | 31.6% | 2.4% |
| 24 | — | — | — | — | — | — | — | — | 88.4% | 7.5% |

Formulation 10 C has a 15.8% coating of ethylcellulose 100/triacetin ( 1/10) applied to the tablet from a 95% ethanol solution.
Formulation 2 was prepared by mixing sodium nitrite with powdered ethylcelluose (Ethocel ® 100) and compressing the blend into tablets. Formulation 5 was prepared by mixing sodium nitrite, powdered ethylcelluose (Ethocel ® 100)and HPMC K100M and compressing the blend into tablets.
Formulations 17 C contains hydroxypropylcellulose (Klucel MF) and has a 8.65% coating of ethylcellulose 100/triacetin (4/1) applied from a chloroform solution.

Simulations of Nitrite Plasma Levels from Controlled Release Formulations

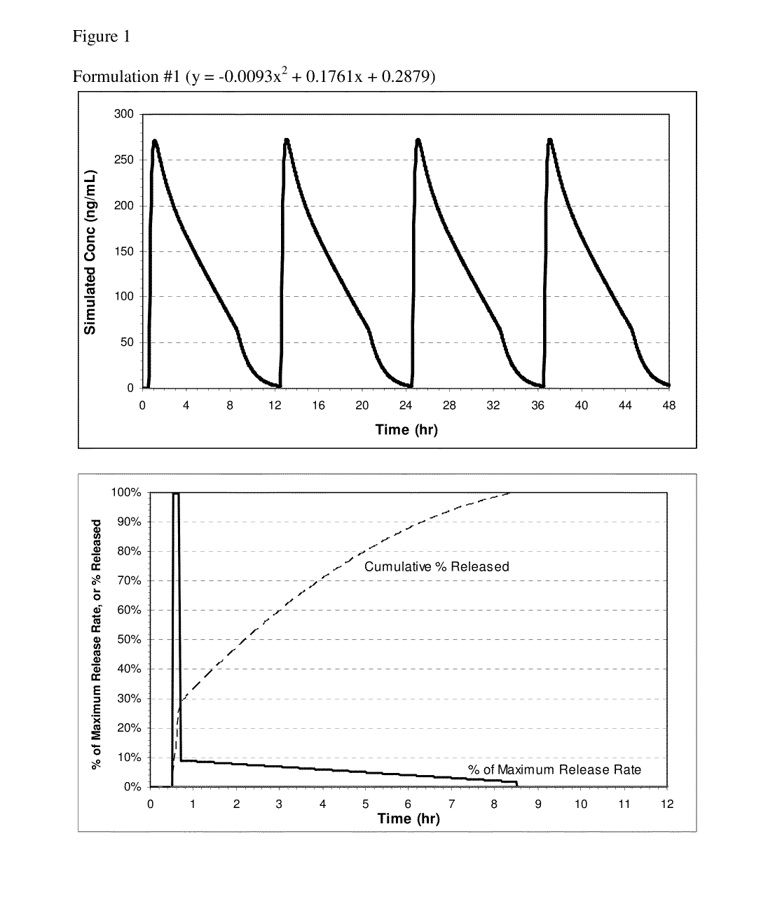

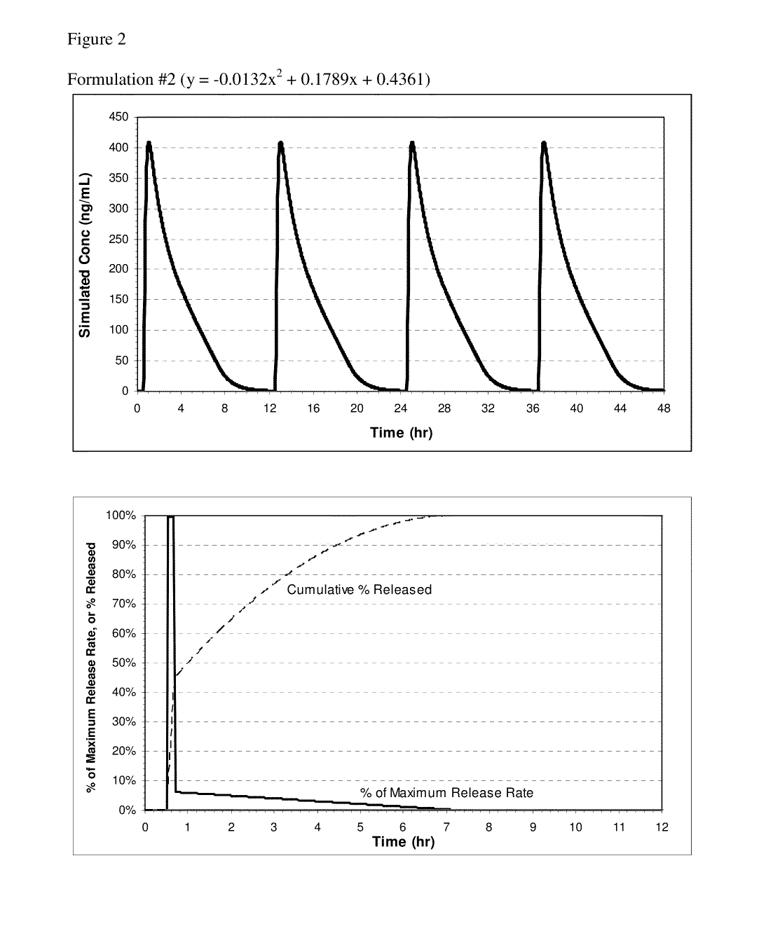

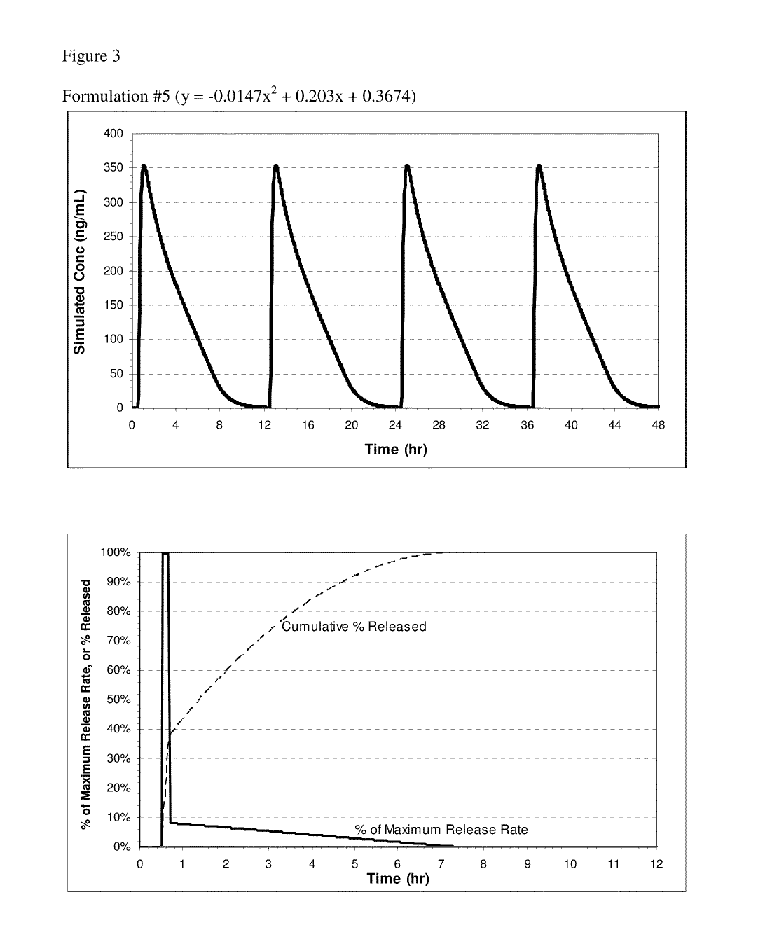

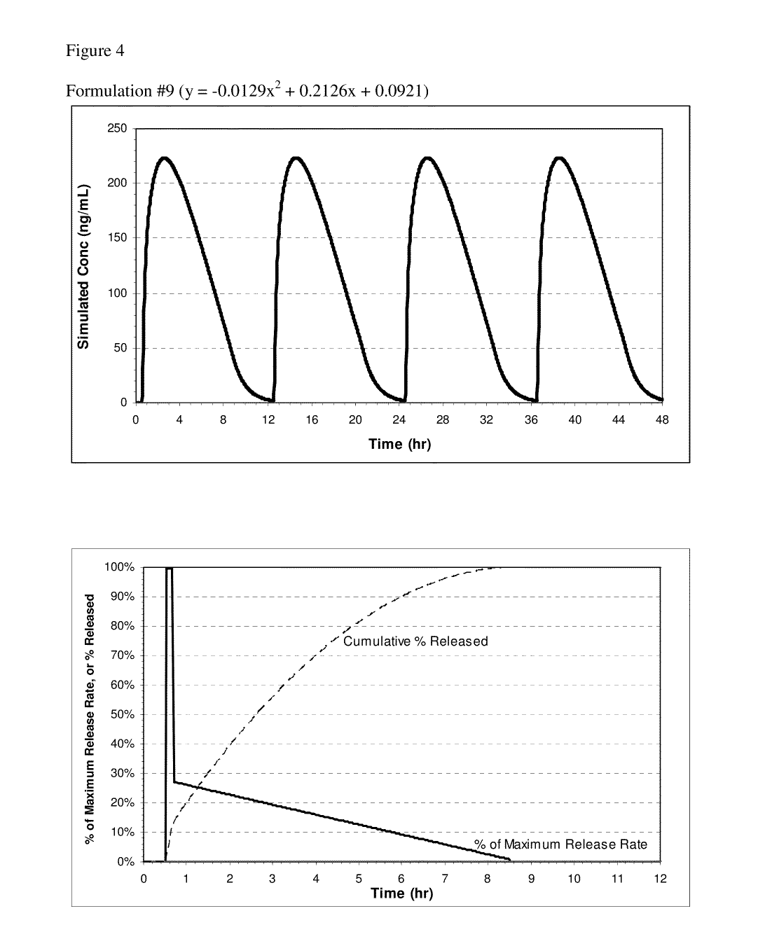

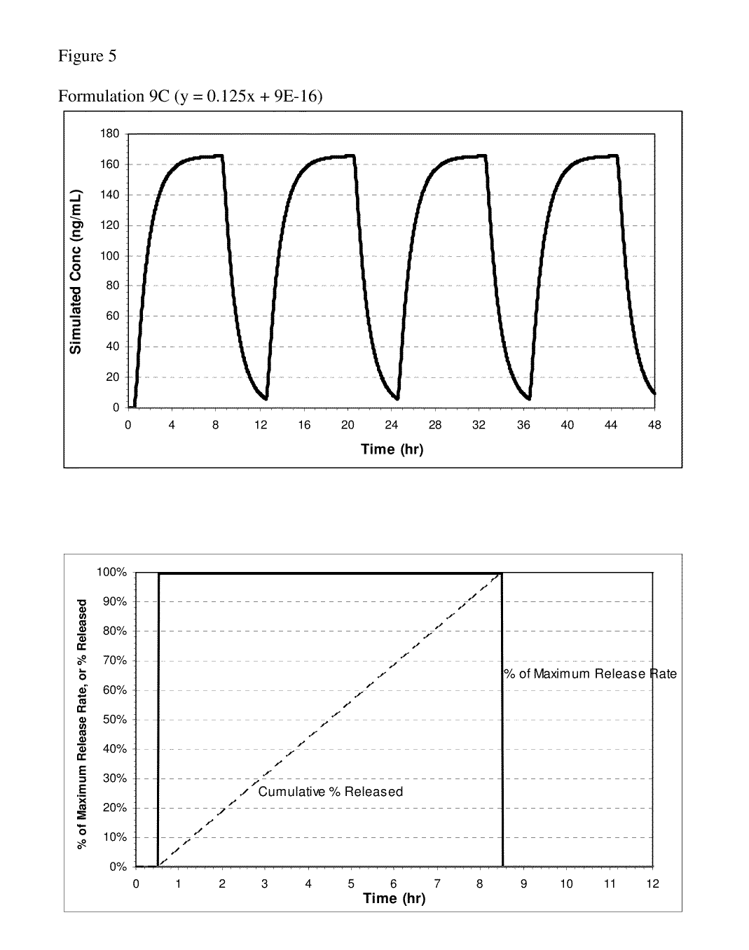

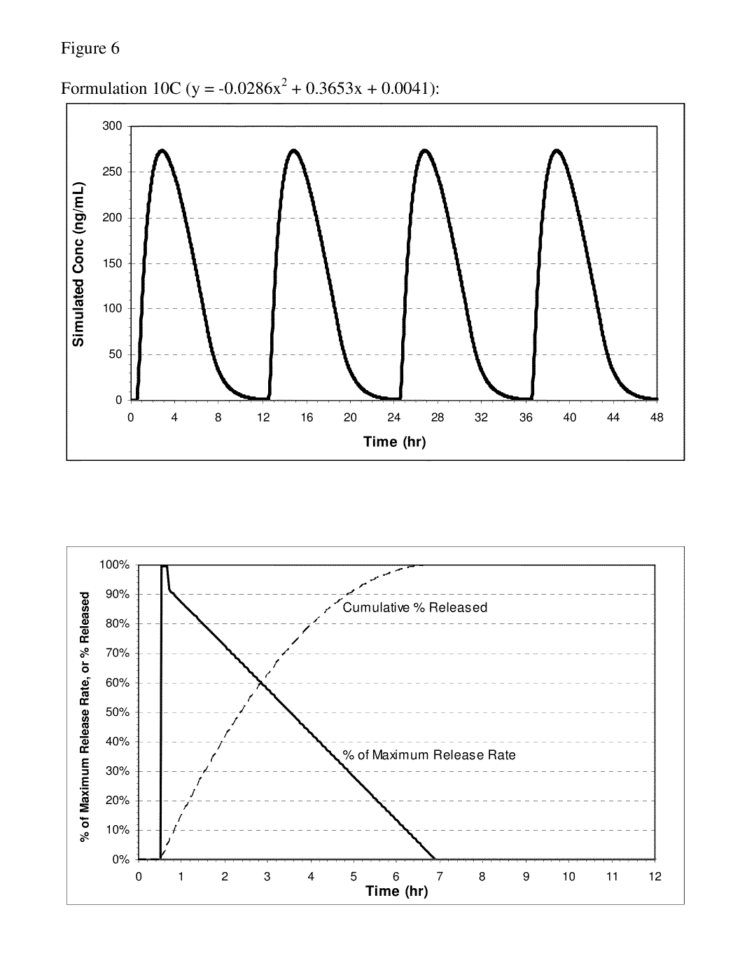

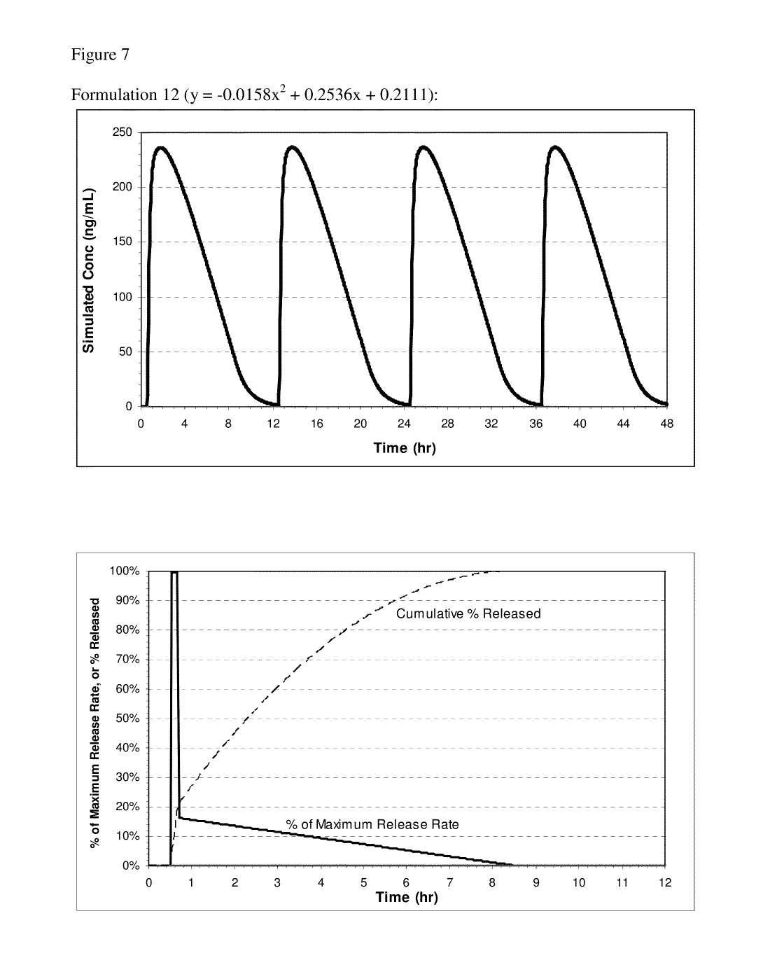

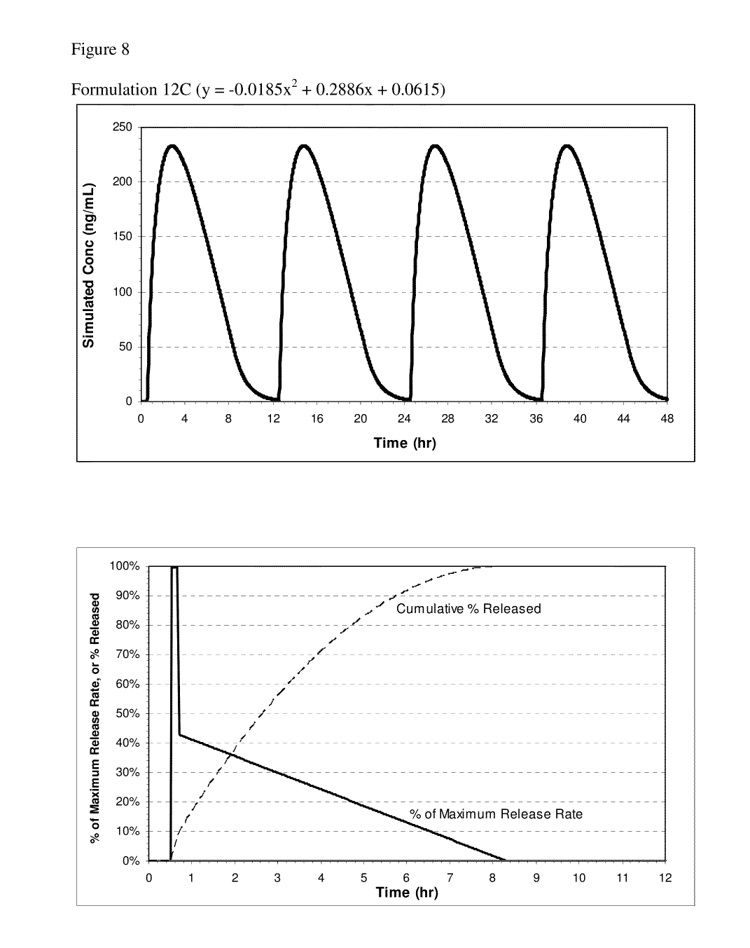

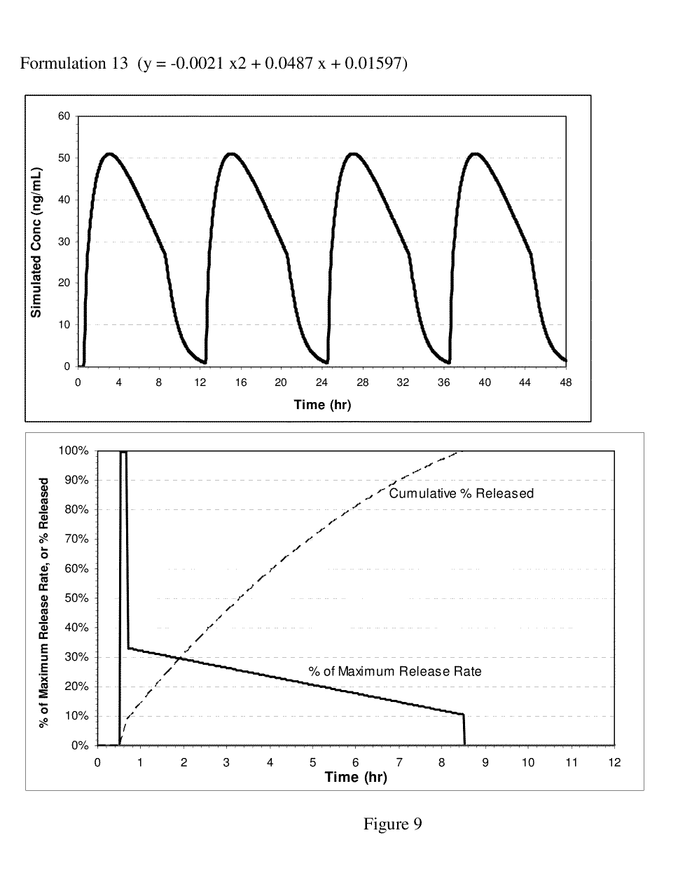

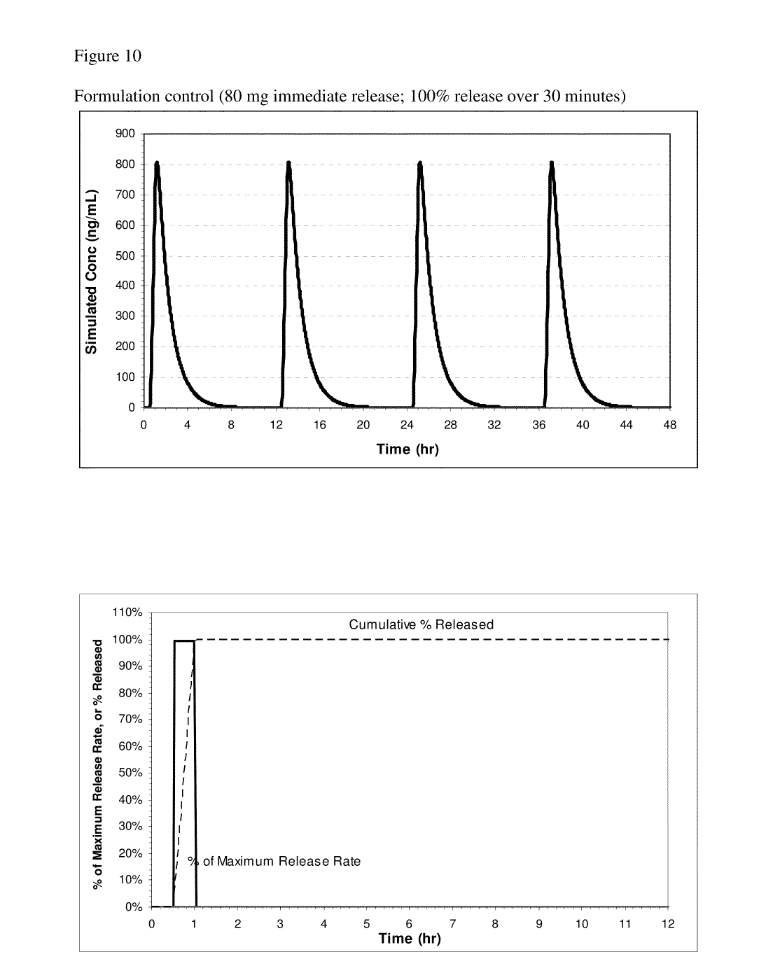

Certain of the above formulations were simulated for determination of their nitrite plasma levels. The simulations assume mid-range pharmacokinetic constants and an 80 mg dose. The assumed PK parameters for NaNO₂ are: half-life=45 minutes; clearance=60.375 L/hr; oral bioavailability=100% (except for formulation 27, which is 27%); lag time between dosing and reaching a pH where the release can occur=0.5 hours. The simulations are for the first two days of twice daily dosing. A concentration of 69 ng/mL is equivalent to 1 μM, and 138 ng/mL is 2 μM. The results are shown in FIGS. 1-10.

For formulations 1, 2, 5, 9, 10C, 12, and 12 C, the equations fit to the profiles had non-zero y-axis intercepts, i.e., at t=0, the % released was some positive number (the constant in the fitted polynomial). For simulation purposes, this was treated as an immediate release component, and that fraction was assumed to be released uniformly over the first 10 minutes after the lag time. Therefore, the release rate profiles show a “spike” in release over that 10 minutes, while the “% released” profile shows a sharp difference in slope between the first 10 minutes and the remainder of the 8 hours of release.

Enteric Coated Capsule Formulations

In some embodiments, the pharmaceutical composition can be formulated as an enteric coated capsule. Tables 8 and 9 provide a formulation for enteric coated capsule formulations.

TABLE 8
Capsule Contents

| Component | Amount (mg/capsule) |
|---|---|
| Capsule Contents | |
| Sodium nitrite, USP | 80 |
| Microcrystalline Cellulose, | 106.5 |
| NF (Avicel ® PH 105) | |
| Blue Food Coloring | 0.5 |
| Size #1 Capsule (Capsugel) | N/A |

TABLE 13
Coating Solution

| Component | Amount | |
|---|---|---|
| Cellacefate, NF | 10 | g |
| (Cellulose Acetate Phthalate) | | |
| Triacetin, USP | 2.2 | mL |
| 95% Ethanol/Acetone (1:1 Volume ratio) | 87.8 | mL |

In this procedure, capsules were prepared by blending sodium nitrite, microcrystalline cellulose, and blue food coloring using standard blending methods for powders. The blended components were manually filled into size #1 capsule shells using small-scale capsule filling equipment. The finished capsules were tested for weight variation and content uniformity to meet compendia requirements for capsules.

The filled capsules were placed in a Procoater holder so that the cap side of each capsule was up. The coating tray was filled with coating solution to within one mm of the top. More coating solution was added to the tray, as needed, after each dip coating step.

The cap side of capsules was dipped into, and slowly removed from, coating solution. Excess coating solution was carefully wiped from the bottom of the capsules so that dried coating was symmetrical on the coating cap. Capsules were placed in a holder on a drying tray for 1 hour. The coating steps were repeated four more times for a total of five coatings.

After the coating was dried, the holder with the capsules was placed on a reversing stand with the cap side down (body side up) and the capsules were pushed into the lowest position with a coating tray cover. The body side of the capsules was dipped into a coating solution and slowly removed from the coating solution. The excess coating solution was carefully wiped from the bottom of the capsules so that the dried coating was symmetrical on the coating body. The capsules and holder was then placed on the drying tray for one hour. The coating steps were repeated four more times for a total of five coatings.

The enteric coated capsules were tested for sodium nitrate release. Uncoated capsules dissolved more than 75% in 0.1N HCl (1 L) in 60 minutes at 37° C. using the USP paddle method at 50 rpm. In 750 mL 0.1N HCl, enteric coated capsules released less than 1% sodium nitrite in 120 minutes at 37° C. using the USP paddle method at 50 rpm. After the pH of the solution was raised to 6.8 with the addition of 250 mL of 0.2 M tribasic sodium phosphate to rhe 750 mL 0.1N HCl solution, the enteric coated capsules released more than 75% sodium nitrite in 60 minutes with 15-16 pancreatin added at 37° C. using the USP paddle method at 50 rpm.

### Rabbit Pharmacokinetic Study

New Zealand rabbits with a weight of 3.0-3.2 kg were used for pharmacokinetic analysis of sustained release sodium nitrite formulations. One milliliter of blood was taken at 14 time points over a six hour period.

Initially, each rabbit was given 31 mg/kg of ketamine with 2 mg/kg of xylazine diluted in sterile normal saline i.m. A second i.m. injection of 0.5 mg/kg of acepromazine was also given at this time. As the rabbits lose consciousness, one ear was shaved with clippers. The area to insert the catheter was cleaned with an alcohol wipe and a 22 gauge iv catheter was inserted into the middle ear artery. A straight injection port was added to seal the end of the catheter. Blood was drawn with a 22 gauge needle and 500 μL of a 1 unit/mL heparin solution was immediately flushed through the catheter. This heparin flush was used after every blood draw.

Following the first blood draw, an 18 Fr gavage tube (36 cm long) was inserted down the esophagous of the rabbit. At the end of the gavage tube, the nitrite capsule is inserted and quickly pushed into the stomach with 15 mL of air. Three formulations were tested: formulations 100A, 200A, and 300A. The gavage tube was then removed and the remaining blood was taken over the next six hours.

The blood draw was equally divided into two 1.5 mL micro centrifuge tubes. 100 μL of plasma nitrite preservation solution was immediately added to one aliquot, while the other aliquot was spun at 5,000 rpm for 2 minutes to separate out plasma that was then combined with 200 μl of plasma nitrite preservation solution. All samples were stored in liquid nitrogen until processing.

The plasma nitrite preservation solution included:

7.85 grams KFeCN+25 mL of PBS=1

66 mg NEM+3 mL of PBS=2

1.5 mL of Nonidet™ P40 (octylphenoxypolyethoxyethanol)=3

1(21 mL)+2(2.5 mL)+3=nitrite preservation solution

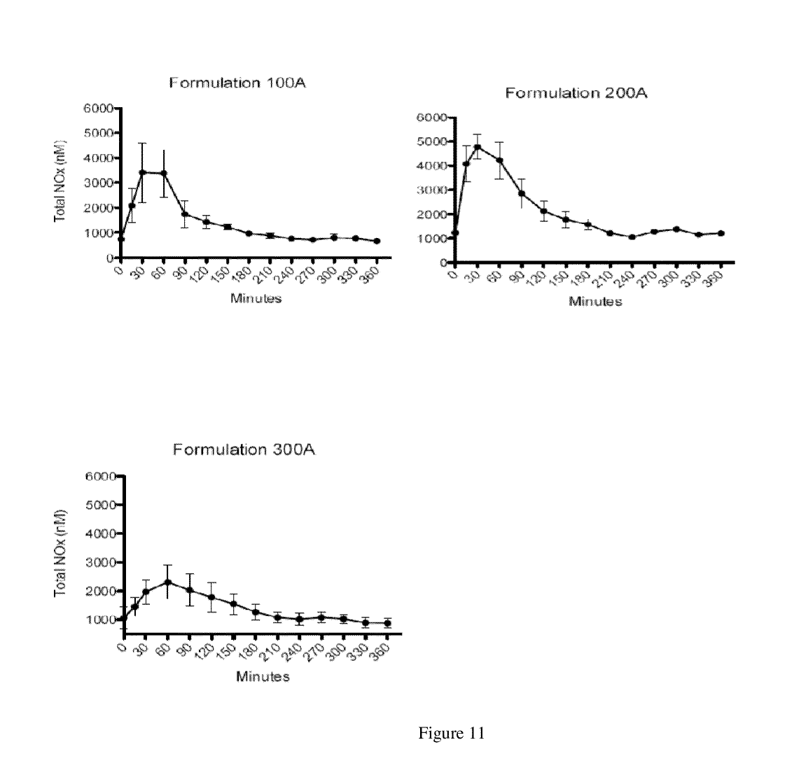

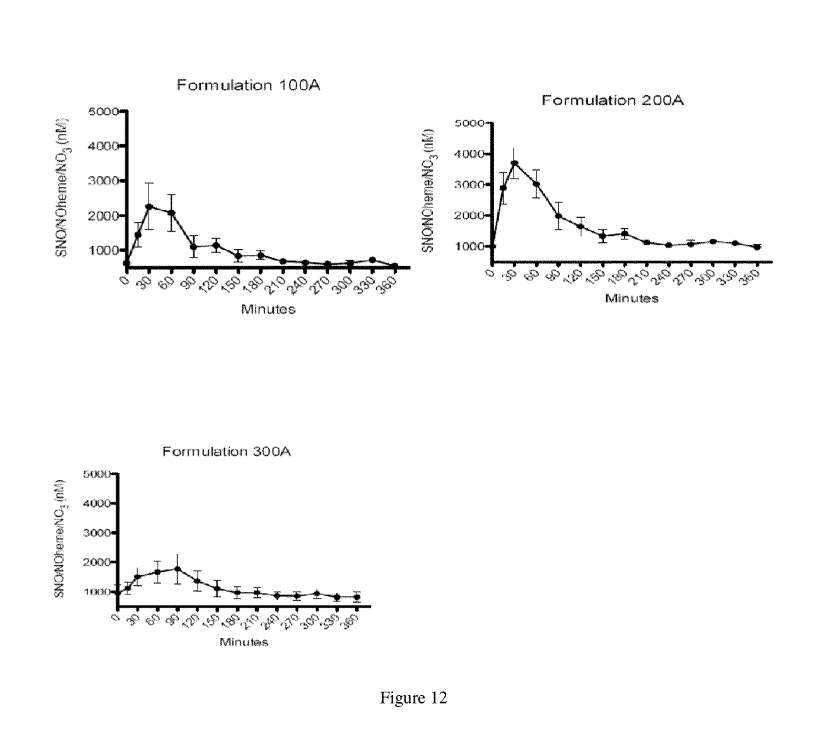

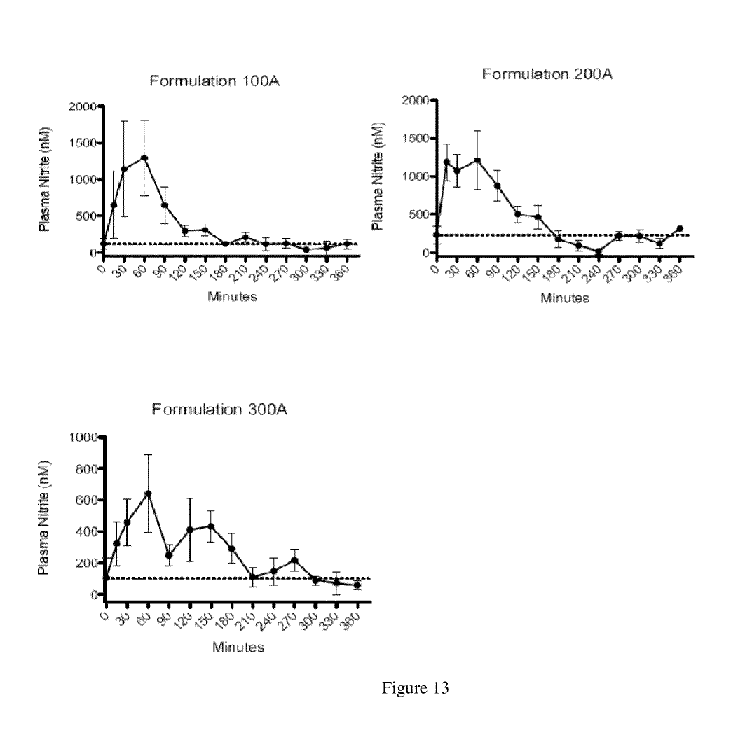

Total NOx in the plasma was calculated as described below. The time courses for each of the three tested formulations are shown in FIG. 11. Additionally, the amount of free nitrite was calculated by treating the samples with 580 mM sulfanilamide in 1N HCl for 15 minutes. This treatment scavenges the free nitrite, leaving behind nitrate, nitrosothiols, nitrosoheme, and nitrosamines. The amount of these remaining components, determined using the method described below, is shown in FIG. 12. When this amount is subtracted from the amount of total NOx, the resulting number reflects the amount of free nitrite (FIG. 13). The data for formulations 200A and 300A represent the mean of five rabbits, while the data for formulation 100A represent the mean of four rabbits, as one rabbit administered the latter formulation experienced a clogging of its arterial catheter during the study.

### Nitric Oxide Chemiluminescence Detection

A Sievers 280i Nitric oxide analyzer (NOA) was used to construct a standard curve of nitrite/NO concentrations and to measure specimen total NOx, nitrosothiols (SNO)+nitrosoheme+nitrate, and free nitrite. To measure nitrite, the purge vessel contained a reducing agent (2 mL sodium iodide in 7 mL glacial acetic acid) to reduce nitrite, nitrate, and nitroso compounds to free nitric oxide. NO gas is then detected in the NOA through a reaction with ozone emitting a photon of light which is detected by the chemiluminescence detector. The amount of NO present was determined by integrating the emission signal over time and calibrated against known amounts of sodium nitrite (0, 0.1, 0.5, 1, 10 and 100 μM) as a source standard for NO. Plasma nitrite was determined by reacting an aliquot of plasma with 580 nM sulfanilamide in 1N HCl for 15 min to scavenge free nitrite. The total amount of free nitrite was determined by subtracting the sulfanilamide value from the total NOx value.

The following list of abbreviations and definitions of terms are used in the examples described hereafter.

| Abbreviations | Term |
|---|---|
| ABI | Ankle Brachial Index |
| ACE | Angiotensin Converting Enzyme |
| ACS | Acute Coronary Syndrome |
| AUC | Area Under Curve |
| AE | Adverse Event |
| BAR | Brachial Arterial Reactivity |
| BID | Twice Daily |
| CBC | Complete Blood Count |
| CFR | Code of Federal Regulations |
| CHF | Congestive Heart Failure |
| CLI | Critical Limb Ischemia |
| CNS | Central Nervous System |
| Cₘₐₓ | Maximum Plasma Drug |
| | Concentration |
| Cₜₐᵤ | Average Drug Concentration over |
| | Dosing Interval |
| CV | Cardiovascular |
| CVD | Cardiovascular Disease |
| DAPI | 4′,6-diamidino-2-phenylindole |
| DBP | Diastolic Blood Pressure |
| DLT | Dose-Limiting Toxicity |
| ECG | Electrocardiogram |
| eCRF | Electronic Case Report Form |
| EDC | Electronic Data Capture |
| eNOS | Endothelial Nitric Oxide Synthase |
| FDA | Food and Drug Administration |
| FMD | Flow-Mediated Vasodilation |
| G6PD | Glucose-6 Phosphate |
| | Dehydrogenase |
| GCP | Good Clinical Practice |
| HbA1c | Hemoglobin A1c |
| IB | Investigator Brochure |
| IC | Intermittent Claudication |
| ICF | Informed Consent Form |
| ICH | International Conference on |
| | Harmonisation |
| IEC | Independent Ethics Committee |
| IL-6 | Interleukin-6 |
| iNOS | Inducible Nitric Oxide Synthase |
| IP | Investigational Product |
| IRB | Institutional Review Board |
| IVRS | Interactive Voice Response |
| | System |
| LOCF | Last Observation Carried Forward |
| MCFA | Medial Circumflex Femoral Artery |
| MDRD | Modification of Diet in Renal |
| | Disease Study |
| MetHb | Methemoglobin |
| MI | Myocardial Infarction |
| nNOS | Neuronal Nitric Oxide Synthase |
| NO | Nitric Oxide |
| NOS | Nitric Oxide Synthase |
| NYHA | New York Heart Association |
| PAD | Peripheral Artery Disease |
| PD | Pharmacodynamic |
| PECAM-1 | Platelet Endothelial Cell Adhesion |
| | Molecule |
| PI | Principal Investigator |
| PK | Pharmacokinetic |
| QoL | Quality of Life |
| RAND 36 | RAND 36-Item Short Form Health |
| | Survey |
| SAE | Serious Adverse Event |
| SBP | Systolic Blood Pressure |
| SD | Standard Deviation |
| SICAM | Soluble Intercellular Adhesion |
| | Molecule |
| SOC | System Organ Class |
| TIA | Transient Ischemic Attack |
| VCAM | Vascular cell adhesion protein |
| VEGF | Vascular endothelial growth factor |
| WIQ | Walking Impairment |
| | Questionnaire |

### Phase 1 Clinical Studies

Study Rationale and Details

The pharmacokinetics, pharmacodynamics, and safety of sodium nitrite have been examined in 8 Phase 1 single-dose and ascending single-dose studies conducted in healthy volunteers. Two studies examined the effect of hypoxia on nitrite-induced vasodilation. One study examined the in vivo conversion of nitrate to nitrite. Description of the clinical studies are provided below.

A Phase 1, single ascending dose study in healthy volunteers was designed to examine the single-dose pharmacokinetics of IV sodium nitrite for 90-130 mg (0.4 mmol/mmol hemoglobin over 10 and 30 minutes), 190-250 mg (0.08 mmol/mmol hemoglobin), and 290-370 mg (0.12 mmol/mmol hemoglobin). Blood samples were analyzed to determine absolute bioavailability and plasma concentrations of nitrite and nitrate as well as hemoglobin (i.e., deoxyhemoglobin, oxyhemoglobin, carbocyhemoglobin, and metHb). Adverse events, blook pressure and heart rate were recorded. Three volunteers (2 females, 1 male) participated in the study, and each volunteer received each single IV dose separated by a washout period of >7 days.

A Phase 1, open-label, 3-way cross-over study in healthy volunteers was designed to examine the single-dose pharmacokinetics of oral and IV sodium nitrite at doses ranging between 140-190 mg (oral; 0.06 mmol/mmol hemoglobin) and 290-380 mg (oral and IV; 0.12 mmol/mmol hemoglobin). Blood samples were analyzed to determine absolute bioavailability and plasma concentrations of nitrite, nitrate, and hemoglobin (i.e., deoxyhemoglobin, oxyhemoglobin, carbocyhemoglobin, and metHb). Nine volunteers (7 females, 2 males) were randomized into the study, and each volunteer received each dose separated by a washout period of ≧7 days

A Phase 1, single ascending dose study in healthy volunteers was designed to examine the pharmacokinetics, safety, and feasibility of 48 hours of IV sodium nitrite administration. Twelve healthy volunteers were intravenously infused with increasing doses of sodium nitrite for 48 hours at doses of 4.2, 8.3, 16.7, 33.4, 66.8, 133.4, 266.9, 445.7, and 533.8 μg/kg/hr. Individual subjects only received one dose. Blood samples were analyzed to determine bioavailability, plasma, and red blood cell concentrations of nitrite, nitrate, and metHb. Clinical data were collected before, during, and after infusion cessation.

A Phase 1, open-label, 4-way cross-over study was designed to examine the single-dose pharmacokinetics of sodium nitrate administered orally via raw and cooked vegetables. Twelve healthy volunteers (6 females, 6 males) were enrolled, and each received the following doses: 365 mg (IV), 564 mg (cooked spinach), 643 mg (cooked beetroot), and 1014 mg (raw lettuce). Blood samples were analyzed to determine absolute bioavailability and plasma nitrate concentrations. The in vivo conversion of nitrate to nitrite was also examined.

A Phase 1, open label, placebo-controlled study in healthy volunteers examined the vasodilatory effects of 2 IV doses of sodium nitrite within forearm vasculature. Eighteen healthy volunteers (9 females, 9 males) were enrolled to receive 75 mg (n=18; 2, 15-minute infusions [±L-NMMA]) and 0.4 mg (n=10; 1, 5-minute infusion). Forearm blood flow and mean arterial pressure were measured. Blood samples were analyzed to determine plasma nitrite concentrations, venous oxygen saturation, pH, iron-nitrosylated hemoglobin, and S-nitroso-hemoglobin concentrations.

A Phase 1, open-label, placebo-controlled, single-dose, dose-escalation study in healthy volunteers at sodium nitrite dose from 0, 7, 14, 28, 55-110 μg/kg/min, then a saline was infused for 180 minutes followed by a final dose of 28 μg/kg/min for 5 minutes. The second cohort (n=15) was randomized to receive a co-infusion of sodium nitrite (0.07, 0.140, 0.350, 0.700, 1.400, 3.500, 7, 14, 28 μg/kg/min) with saline (0.9%, n=5), oxypurinol (600 μg/min, n=5), or ascorbin acid (24 mg/min, n=5). A Phase I, open-label, single-dose, dose-escalation study enrolled 40 healthy volunteers (6 females, 27 males) into 2 cohorts. The first cohort (n=26) received 30-minute infusions of sodium nitrite at 40, 100, 314, 784 mmol/min and 3.14 and 7.84 μmol/min Volunteers in the second cohort received 7.84 μmol/min (n=7) or 314 nmol/min (n=7) at ambient and 12% oxygen concentrations.

A Phase 1, single-blind, placebo-controlled single-dose study was designed to examine hypoxic effects on nitrate-induced vasodilation. Eighteen healthy male volunteers received IV sodium nitrite (n=12; 1 μmol/min for 30 minutes) or placebo (n=6) after stabilizing at 12% oxygen concentration. Half of the test group (n=6) also received sodium nitrite at 21% oxygen concentration.

A Phase 1, open-label, single dose crossover study of 80 mg sodium nitrite immediate release formulation and 80 mg enteric coated sodium nitrite formulation in patients with diabetes and PAD have also been performed. Table 14 shows the details of the study on 12 diabetic patients with PAD.

TABLE 14
Phase 1 Single-dose study in Diabetic Patients with PAD

| | |
|---|---|
| Baseline Nitrite Levels | 0.521 (0.003-2.580) |
| Peak Nitrite Levels | 4.630 (1.159-10.172) |
| Baseline MetHb Levels | 0.291 (0.1-0.5) |
| Peak MetHb Levels | 0.375 (0.2-0.6) |
| Adverse events | 9: none |
| | 1: flushing |
| | 1: headache |
| | 1: headache, nausea, hot flash |

Clinical Pharmacology

### Single, Ascending-Dose Pharmacokinetics of Intravenous Sodium Nitrite in Healthy Adults

Single IV doses of sodium nitrite resulted in maximum plasma nitrite concentrations immediately following infusion between 0.9 and 4.8 mg/kg. With the 0.04 mmol/mmol hemoglobin dose infused over 10 minutes, maximal plasma nitrite concentrations of 1.4-2.8 mg/kg were observed. Extending the 0.04 mmol/mmol hemoglobin infusion over 30 minutes resulted in maximum plasma nitrite concentrations of 0.9-1.3 mg/kg. Doubling of the dose over 30 minutes resulted in maximum plasma nitrite concentrations of 2.1-3.3 mg/kg, and a tripling of the dose infused over 30 minutes resulted in maximum plasma nitrite concentrations of 3.3-4.8 mg/kg. The elimination half-life of plasma nitrite concentration ranged between 0.48 and 0.60 hours. The AUC increased linearly with increasing sodium nitrite dose. Adjusted to a standard dose of 220 mg sodium nitrite, the AUC ranged between 7.846 and 2.616 mg*hr/L among the 3 healthy volunteers. The volume of distribution for the 2 highest doses was estimated to range between 60 and 77 liters at the 0.08 mmol/mmol hemoglobin dose and between 66 and 83 liters at the 0.12 mmol/mmol hemoglobin dose.

Overall, the drug was well tolerated. Systolic and diastolic blood pressures decreased with similar magnitude (maximum decrease of approximately −13/15 mmHg) across the doses with no dose-dependent effect observed. A compensatory increase in heart rate of 11-14 bpm was generally observed. No effect on liver enzymes was observed. No serious adverse effects were observed during any of the dosing procedures. All adverse effects reported by volunteers were of mild intensity. Dizziness, headache, and head discomfort were reported which are known to be related to sodium nitrite administration. One subject experienced eye accommodation disorder. This adverse effect was unexpected and considered definitely related to sodium nitrite.

During IV dosing, metHb increased gradually and continued for some time after infusion was stopped. Methemoglobin at the lowest dose ranged between 2.0-3.2%. Doubling of the dose resulted in metHb of 6.5-7.7% and tripling the dose resulted in metHb levels of 10.6-11.0%. The time to reach the maximum percentage of metHb (Tₘₐₓ) observed at the highest dose. The elimination half-life of metHb across doses ranged between 0.86 and 1.30 hours.

In summary, ascending, single IV doses of up to 0.12 mmol/mmol hemoglobin (290-370 mg sodium nitrite) reached peak plasma concentrations of 4.8 mg/kg, has a terminal half-life of 30 minutes, and induced approximately 10.8% metHb in the blood. Infusion of 290-370 mg sodium nitrite was defined as the maximum tolerated dose without considerable adverse effects. Sodium nitrite lowered blood pressure, and resulted in a compensatory increase in heart rate.

### Single-Dose Study of Oral and Intravenous Sodium Nitrite in Healthy Adults

Absorption of inorganic nitrite (NO₂⁻) from the gastrointestinal tract was rapid following a single oral dose. Baseline plasma nitrite concentrations (n=8) were below the lower limit of quantification (0.1 mg/kg), and after reaching Cₘₐₓ at approximately 1.6 hours (140-190 mg, oral) and 3.1 hours (290-380 mg, oral and IV), nitrite demonstrated an elimination half-life of 21-35 minutes across the doses. The IV and oral doses between 290-380 mg demonstrated nearly identical concentration-time curves. Absolute bioavailability of nitrite was 70-110% (low oral dose range) and 73-120% (high oral dose range). Plasma nitrite rapidly reacted with hemoglobin to form nitrate and metHb, but the percent plasma metHb (IV: 8.4-12%; low oral: 3.4-4.5%; high oral: 7.7-11%) remained below clinically toxic levels (less than 15%). Maximal plasma metHb concentrations were reached in 0.8 hours (low oral dose) and 1-1.2 hours (IV and high oral dose).

Single doses of sodium nitrite (140-190 mg and 290-380 mg) were generally well tolerated, and only minor adverse effects were reported. Three volunteers reported nausea with oral and IV administration (290-380 mg, oral and IV). Headaches were reported by 5 (290-380 mg, IV), 4 (290-380 mg, oral), and 4 (140-190 mg, oral) volunteers. For all reported cases, nauseas subsided within 30 minutes, and headache subsided within 23 hours.

Single, Ascending-Dose Study of 48-hour Intravenous Sodium Nitrite in Healthy Adults

Forty-eight hour infusions of IV sodium nitrite were conducted in healthy volunteers (n=12) with increasing doses (from 4.2 to 533.8 μg/hr). The peak blood nitrite concentration in subjects treated with 266.9 μg/kg/hr was 1.1 μgM, and the peak blood nitrite concentration in subjects treated with 445.7-533.8 μg/kg/hr was 3.4 μM. Analysis of nitrite AUC relative to the corresponding dose indicated systemic exposure to nitrite in plasma and whole blood increased less than proportionately with increasing dose. The non-linear pharmacokinetic appeared to be related to increased clearance from the body at higher dose. The non-linear pharmacokinetic appeared to be related to increased clearance from the body at higher doses. The volume of distribution also appeared to increase with higher doses. The mean elimination half-life of plasma sodium nitrite at maximal tolerated dose was 43.1 minutes and for whole blood was 51.4 minutes.

The maximal tolerated dose for 48-hr IV infusion of sodium nitrite was established at 266.9 μg/kg/hr, and dose limiting toxicity was determined at 445.7 μg/kg/hr. There was no evidence of toxicity at the 266.9 μg/kg/hr dose. No changes in mean arterial pressure were observed at doses up to 266.9 μg/kg/hr, and the peak metHb observed at these dosing levels was 1.8%. MetHb levels plateaued within 30 minutes of infusion and remained elevated during the infusion. MetHb returned to baseline levels in approximately 30 minutes after stopping the sodium nitrite infusion. IN subjects treated with 533.8 and 445.7 μg/kg/hr, mean arterial pressure decreased by 15 mmHg and 20 mmHg, respectively. Peak metHb exceeded 5% in one subject (445.7 μg/kg/hr). In all instances of dose limiting toxicity, the subjects were asymptomatic, the effects were transient, and resolved within 12 hours. All other laboratory results remained unaffected by the sodium nitrite infusion with no changes observed in hematological (other than metHg), metabolic, liver, or kidney function tests.

Single-Dose Bioavailability Study of Sodium Nitrate from Raw or Cooked Vegetables

Inorganic nitrate (NO₃⁻) was efficiently absorbed following a single oral dose of raw and cooked vegetables. Plasma nitrite concentrations were unchanged following treatment. Volunteers (n=12) received IV infusion of 500 mg NaNO₃ (365 mg NO₃⁻), 300 g cooked spinach (564 mg NO₃⁻), 300 g raw lettuce (1013 mg NO₃⁻), and 300 g cooked beetroot (643 mg NO₃⁻). Baseline plasma nitrate concentrations ranged between 1.0-5.3 mg/kg. Maximal plasma concentrations were achieved in 30 minutes and 1.5-1.8 hours following IV and vegetable doses, respectively. Absolute bioavailability of nitrate was 90-106% from the consumed vegetables. The in vivo conversion of nitrate to nitrite was negligible given that plasma nitrite concentrations remained below the lower limit of quantification (0.2 mg/kg) in most samples.

### Single-Dose Study of Intravenous Sodium Nitrite in Healthy Volunteers

Vasoactivity of infused sodium nitrite was characterized at supra- and normo-physiologic plasma nitrite concentrations. Sodium nitrite infusion (n=18) produced a regional IV sodium nitrite concentration of 221.82±57.59 μM as compared to the contralateral (systemic) IV sodium nitrite concentration of 16 μM. Forearm blood flow increased 175%, and statistically significant increases were observed in venous hemoglobin oxygen saturation, pO₂, and pH. Forearm blood flow was additionally increased with forearm exercise. Systemic mean blood pressure was reduced approximately 7 mmHg. Increased plama nitrite concentrations were associated with persistent forearm vasodilation and reduced systemic blood pressure at 1 hour post dose. Ipsilateral co-infusion of sodium nitrite and NO synthase inhibitor resulted in vasodilatory effects similar to sodium nitrite alone.

Physiologically relevant vascular nitrite concentrations (400 nmol/mL) were evaluated (n=10). After 5 minutes (1 mL/min), mean venous nitrate concentrations increased from 176±17 nM to 2564±462 nM. Forearm blood flow increased at rest and during NO synthase inhibition with or without exercise.

### Ascending Single-Dose Study of Intravenous Sodium Nitrite in Healthy Volunteers

Following sodium nitrite infusion (0, 7, 14, 28, and 55, to 110 μg/kg/min), nitrite metabolism was measured over 180 minutes. Baseline plasma nitrite was 0.13±0.049 μmol/L, increasing to 26.1±6.1 μmol/L during infusion. In parallel, intra-erythrocyte nitrite concentration increased from 0.29±0.06 to 34.9±6.7 μmol. Using a 2 compartment model, the volume of distribution was 25.2±10.3 L. Mean clearance was 0.948±0.423 L/min, and inter-compartmental clearance was 0.665±0.424 L/min. Terminal half-life was 42.1±10.2 minutes.

Dose-dependent increase in forearm blood flow was observed between 2.8±0.2 and 12.3±1.4 mL/min/100 mL. Forearm blood flow increases correlated with ipsilateral increases of whole blood nitrite concentration, but the vasodilatory effect was saturated above 300 μmol/L nitrate. The ED50 of nitrite-induced vasodilation was 18.6 μg/kg/min.

Ipsilateral forearm blood flow (n=5) increased rapidly. At 7 μg/kg/min, ipsilateral blood flow increased significantly after 60 seconds, whereas the higher dose of 28 μg/kg/min resulted n increased ipsilateral blood flow after 15 seconds. Contralateral blood flow (n=5) increased significantly after one minute of nitrate infusion (28 μg/kg/min) and showed whole blood nitrite concentration of 0.65±0.15 to 4.6±1.1 μmo/L at the time of contralateral effect. Mean arterial pressure decreased (97±1.7 to 86±2.2 mmHg, p<0.001) and heart rate increased (68±3 to 76±4 bpm) in association with increased blood nitrite and NO formation. Mean arterial pressure and nitrate infusion rate were inversely related (r=−0.47, p=0.0088; n=30 measurements in 5 subjects). Decreased mean arterial pressure persisted for 120 minutes after cessation of nitrite infusion with a gradual return to baseline pressure.

### Ascending Single-Dose Study of Intravenous Sodium Nitrite During Hypoxia

In the forearm vasculature, hypoxia modulated vasodilation resulting in more potent arterial dilation during hypoxia as compared to normoxia. At the 3 highest study doses of sodium nitrite infusion (784 nmol/min, 3.14 μmol/min, and 7.84 μmol/min), venous tone showed large decreases within the first 5 minutes (n=26). Peak venodilation which occurred at 20 minutes (3.14 and 7.84 μmol/min) was 20.6±4.2% (p<0.05) and 35.8±7.5% (p<0.005). The ratio of forearm blood flow increased at 3.14 and 7.84 μmol/min (1.8±0.3 and 1.6±0.2, respectively [p<0.05]).

At 7.84 μmol/min nitrite, venodilation was comparable between room air and 12% oxygen (n=7). However, forearm blood flow was increased during hypoxia as compared to normoxia (p<0.05). A similar effect (n=7) was obsesrved at a physiologically relevant nitrite concentration (314 nmol/min)

### Single-Dose Study of Low-Dose Intravenous Sodium Nitrite During Hypoxia

A low dose of infused sodium nitrite (1 mol/min for 30 minutes) had significant and prolonged vasodilatory effects on hypoxic pulmonary circulation and pulmonary arterial pressure was reduced 12-17%. Forearm blood flow and 3 echo surrogate indexes of pulmonary arterial pressure were measured at 12% and 21% oxygen during sodium nitrite infusion (n=18).

Forearm blood flow increased with nitrite administration during hypoxia (peak, 3.0±0.2 mL/100 mL/min, p<0.01) but not when nitrite was infused during normoxia or when saline was infused during hypoxia. Forearm blood flow had returned to baseline ≦1 hour post dose. Blood flow increases correlated with peak plasma nitrite concentration during hypoxia (Pearson r=0.31, p=0.002), whereas blood flow and plasma nitrite concentration were unrelated during normoxia (Pearson r=0.03, p=0.86).

During hypoxia, surrogate indexes of pulmonary arterial pressure (pulmonary arterial systolic pressure [PASP]; pulmonary acceleration time [PAT]; isovolumetric relaxation time [IVRT]) indicated that increased pressure was secondary to hypoxi pulmonary arterial vasoconstriction. All 3 indexes increase with hypoxia alone, and decreased with nitrite treatment during hypoxia.

Single-Dose Study of Two Formulations of Sodium Nitrite in Diabetic Patients with PAD

In general, 80 mg dose of either sodium nitrite immediate release or enteric coated formulation was safe and well tolerated. Minimal effects on blood pressure were observed and no effect on heart rate was observed. The immediate release formulation quickly increased plasma nitrite within 30 minutes. The enteric coated formulation gradually increased plasma nitrite. Moreover, 80 mg immediate release formulation achieved blood levels anticipated to be therapeutic.

Safety

Humans are exposed to nitrite in their diet, environment, work, and medications. Sodium nitrite is included as an approved therapy for cyanide toxicity. Details regarding the use of IV sodium nitrite can be found in the Cyanide Antidote Kit product label.

Overall, sodium nitrite in clinical trials has been well tolerated. No clinically significant changes were seen in laboratory parameters, with the exception of methemoglobinemia. These abnormalities were incidental and considered to be of no clinical significance by the investigator.

Sodium nitrite lowered SDP and DBP and was associated with compensatory increase in pulse rate. Some individual abnormalities were noted, but such observations were considered to be of minor clinical significance by the investigator.

Acute nitrite toxicity is the result of excessive vasodilation nad hypotension and the development of methemoglbinemia. Methemoglobinemia is the most frequent adverse finding observed with both oral and IV sodium nitrite administration. The therapeutic dose of sodium nitrite for cyanide toxicity is 300 mg IV over 4 minutes, and levels of up to 20% themoglobinemia have been reported in adults at this dose. The pediatric dose is 0.33 mL/kg of a 3% solution at 2.5 mL/min to a maximum dose of 10 mL. Single oral and IV doses of sodium nitrite of up to 380 mg have been administered to humans with a maximum tolerated dose defined as 380 mg sodium nitrite.

Adverse events resulting from intake of nitrites include hypotension, dizziness, syncope, giddiness, cerebral ischemia, headache, reflex tachycardia, increased intraocular pressure, confusion, nausea, vomiting, methemoglobinemia, hemolysis, seizures, myocardial ischemia, coma, cardiovascular collapse, and asphyxia, and can be fatal in overdose. For reported adverse events in clinical trials, most adverse events subsided between 30 minutes and 23 hours. Deaths by case report have been reported with sodium nitrite as accidental poisoning and as a complication of the treatment for cyanide toxicity.

Methemoglobinemia is the most frequent adverse even observed with sodium nitrite administration. The level of metHb increases with increasing dose. Methemoglobin levels of up to 11 and 12% have been observed in clinical trials following maximum tolerated oral doses and IV administration of 380 mg sodium nitrite, respectively. The elimination half life of metHb in clinical studies ranged between 0.86 and 1.30 hours. Hemolytic anemia may also occur acutely after exposure to nitrites.

The acceptable daily intake of nitrite in people over the age of 6 months is 0.4 mg/kg. Overdose of sodium nitrite results in methemoglobinemia. The minimal toxic dose of nitrite varies among individuals. Symptoms of methemoglobinemia may be seen at blood methaemoglobin concentrations of 15%, but symptoms do not usually appear until the blood methaemoglobin concentration reaches 30 to 40%. Methemoglobin levels of 70% or greater are likely to be fatal. The symptoms of methemoglobinemia include cyanosis, headache, unusual tiredness or weakness, tachycardia, shortness of breath, extreme dizziness or fainting, and coma. Cardiovascular collapse, convulsions, and death may occur after sodium nitrite overdose. The mean lethal oral dose of sodium nitrite in adults is approximately 1 g if no treatment is received, although survival after this dose has been reported.

Sodium nitrite is reported to be incompatible with the following: acetanilide, antipyrine, caffeine, citrate, chlorates, hypophosphites, iodides, mercury salts, morphine, oxidizing agents, permanganate, phenazone, sulfites, tannic acid, and vegetable astringent decoctions, infusions, or tinctures. Moreover, severe hypotension has been reported when sildenafil is combined with organic nitrates. It is not known whether a smilar reaction occurs with nitrites; however, it is possible that combined use would produce increase cGMP levels. Concomitant use is therefore considered contraindicated.

### Phase 2a Clinical Studies

Study Rationale and Details

Sodium nitrite was investigated as a new therapy for improving function in subjects with PAD. The overall goal of this dose-ranging study was to evaluate the safety, pharmacokinetics, tolerability, and potential biological activity of multiple doses of oral sodium nitrite in subjects with PAD. As described in detail above, the primary pathophysiology of PAD is related to the limitation in blood flow of the lower extremities, resulting in limited exercise tolerance and decreased quality of life. A common feature of PAD is endothelial dysfunction, decreased NO bioavailability, and depletion of NO stores, a finding that may be compounded when PAD and metabolic diseases, such as diabetes, coexist. Sodium nitrite is an inorganic salt that is found and metabolized in vivo. At physiological concentrations, sodium nitrite is known to cause vasodilation.

The primary objective of this early stage clinical study was to evaluate the safety and tolerability of multiple doses of twice daily 40 mg and 80 mg sodium nitrite compared with placebo over a 10 week treatment period. The secondary objective of this study was to evaluate the pharmacokinetics of sodium nitrite and to demonstrate the pharmacodynamic effect of sodium nitrite on measures of biologic activity and functional measures of walking distance and claudication symptoms. Finally, the relationship between doses, plasma concentration of sodium nitrite, and pharmacodynamic effects were characterized and evaluated. In this study, multiple assessments of biological activity and ambulatory function were made during standardized tests of arterial reactivity and claudication-limited exercise. The pharmacodynamic assessments included: brachial artery flow-mediated vasodilation (FMD), six-minute walk test, selected biomarkers of interest, quality of life questionnaires (WIQ & RAND 36).

The primary endpoints included: clinical safety and tolerability data including spontaneous AE reporting, ECGs, vital signs, nursing/physician observation, and clinical laboratory values. The secondary endpoints included flow-mediated vasodilation responses, maximal distance covered during a six-minute walk test, plasma pharmacokinetics (including but not limited to AUC, Cₘₐₓ, Cₜₐᵤ) of sodium nitrite and the relationship to the pharmacodynamic assessments performed in this study, and quality of life (WIQ & RAND 36). Furthermore, exploratory pharmacodynamic/biomarker endpoints included changes in markers of inflammation, oxidative stress, metabolic function, angiogenesis, or other markers of atherosclerotic disease, as data permitted (e.g. sodium nitrite, nitrite, nitrate, soluble intercellular adhesion molecule (SICAM), Vascular cell adhesion protein (VCAM), F2-isoprostanes and Interleukin-6 (IL-6)).

The trial type was a randomized, double-blind, placebo-controlled, dose ranging, parallel design multiple dosing study targeted on subjects with PAD. Subjects were at least 35 years of age, but not greater than 85 years of age. If the subject experienced claudication, the subjects also had a 1 month history of stable PAD symptoms. Subjects were assigned to either the placebo or sodium nitrite treatment group in accordance with the randomization schedule generated prior to the start of the study. Subjects were randomized into the study by means of an interactive web response system (IWRS) through electronic data capture (EDC) to receive one of the treatment regimens of either placebo, 40 mg BID or 80 mg BID. As this was a double-blind study, subjects, investigators, and site staff were blinded. TheraVasc and CPC were also blinded. In the case of a medical emergency or in the event of a serious medical condition, when knowledge of the investigational product was essential for the clinical management or welfare of the subject, an investigator or other physician managing the subject could unblind that subject's treatment code. The investigator made every effort to contact the CPC Medical Monitor before unblinding to discuss options. If the blind was broken for any reason and the investigator was unable to contact CPC prior to unblinding, the investigator must notify CPC as soon as possible following the unblinding incident without revealing the subject's study treatment assignment, unless the information was important to the safety of subjects remaining in the study. In addition, the investigator would record the date and reason for revealing the blinded treatment assignment for that subject in the appropriate data collection tool. If an expedited regulatory report to one or more regulatory agencies was required, the report identified the subject's treatment assignment. When applicable, a copy of the regulatory report was sent to investigators in accordance with relevant regulations, CPC policy, or both.

The Investigational Product (IP)

The compositions of the invention comprises sodium nitrite (NaNO₂), an inorganic salt used as a color fixative and preservative in meats and fish. It can be produced from nitrates in ingested food by bacteria in the gastrointestinal tract. It is also used in manufacturing diazo dyes, nitroso compounds, and other organic compounds; in dyeing and printing textile fabrics and bleaching fibers; in photography; as a laboratory reagent and a corrosion inhibitor; in metal coatings for phosphatizing and detinning; and in the manufacture of rubber chemicals. Sodium nitrite has also been used in human and veterinary medicine as a vasodilator, an intestinal relaxant, and an antidote for cyanide poisoning.

Capsules of sodium nitrite at dose strength of 40 mg and 80 mg per capsule which were to be stored at controlled room temperature (20-25° C., 68-77° F.). Avoid high humidity and excessive heat above 40° C. (104° F.). Matching placebo capsules were also supplied and stored at controlled room temperature. TV1001 was supplied in 50 count bottles dispensed in accordance with the visit schedule described in Table 15. IP was stored under secure conditions. Bilcare, Global Clinical Supplies labeled, stored and distributed the sodium nitrite and matching placebo. IP was assigned and administered as described below. Table 16 describes details of the study drug.

TABLE 15
Schedule of Assessments

| | Visit Name | | | | | | |
|---|---|---|---|---|---|---|---|
| | | Visit 1 | | | Safety | | |
| | Screening | Randomization | Visit 2 | Visit 3 | Visit¹ | Visit 4 | Visit 5 |
| Timing (days) | −21 to −14 | Day 0 | Day 1 | Day 4 | Day 7 | Day 14 | Day 28 |
| | days | | | | | | |
| Allowance Variance | | | ±4 hours | ±1 day | ±1 day | ±2 days | =2 days |
| Informed Consent | X | | | | | | |
| Demographics | X | | | | | | |
| Medical and Medication | X | X | | | | | |
| History | | | | | | | |
| Physical Examination | X | | | | | | |
| Vital Signs² | X | X | X | X | X | X | X |
| 12-Lead ECG | | X | | | | | X |
| Clinical Safety Labs | X | | X | X | | X | X |
| Met-Hb Labs³ | | | X | X | X | X | X |
| PK Sample | | | X | X | | X | X |
| PK and Met-Hb over 7 time | | X | | | | | |
| points³ | | | | | | | |
| Urine Pregnancy Test | X | X | | | | X | |
| PD Biomarkers | | X | | | | | |
| Ankle Brachial Index (ABI) | X | | | | | | |
| FMD⁴ | | X | | | | | |
| OoL (WIQ, RAND 36) | | X | | | | | |
| Six Minute Walk Test | | X | | | | | |
| Study Medication | | X | | | | X | X |
| Dispensed | | | | | | | |
| Adverse Events | | X | X | X | X | X | X |
| Concomitant Medications | | X | X | X | X | X | X |
| Evaluate | X | X | | | | | |
| Inclusion/Exclusion Criteria | | | | | | | |
| Evaluate Study Stopping | | X | X | X | X | X | X |
| Criteria | | | | | | | |

| | Visit Name | | | | | | | |
|---|---|---|---|---|---|---|---|---|
| | Phone | Phone | | | Safety | Termination | Follow-up | Early |
| | Call 1 | Call 2 | Visit 6 | Visit 7 | Visit¹ | Visit 8 | Phone call | Term |
| Timing (days) | Day 42 | Day 56 | Day 70 | Day 71 | Day | 6 Days after | 7 Days after | n/a |
| | | | | | 71 + 1 | V7 | V8 or ET | |
| Allowance Variance | ±2 days | =2 days | ±2 days | −1 day | −1 | =1 days | ±1 days | n/a |
| | | | | | day | | | |
| Informed Consent | | | | | | | | |
| Demographics | | | | | | | | |
| Medical and Medication | | | | | | | | |
| History | | | | | | | | |
| Physical Examination | | | | | | X | | X |
| Vital Signs² | | | X | X | X | X | | X |
| 12-Lead ECG | | | | | | X | | X |
| Clinical Safety Labs | | | X | X | | X | | X |
| Met-Hb Labs³ | | | X | X | X | X | | X |
| PK Sample | | | X | X | | X | | X |
| PK and Met-Hb over 7 time | | | | | | | | |
| points³ | | | | | | | | |
| Urine Pregnancy Test | | | | | | X | | X |
| PD Biomarkers | | | X | | | | | X |
| Ankle Brachial Index (ABI) | | | | | | | | |
| FMD⁴ | | | X | | | | | X |
| OoL (WIQ, RAND 36) | | | X | | | | | X |
| Six Minute Walk Test | | | X | | | | | X |
| Study Medication | | | | X | | | | |
| Dispensed | | | | | | | | |
| Adverse Events | X | X | X | X | X | X | X | X |
| Concomitant Medications | X | X | X | X | X | X | X | X |
| Evaluate | | | | | | | | |
| Inclusion/Exclusion Criteria | | | | | | | | |
| Evaluate Study Stopping | | | X | X | X | | | |
| Criteria | | | | | | | | |

¹This visit is only required if Met-Hb is 8% or greater.
²Vital signs are supine prior to first dose of IP and postural after first dose.
³Repeated blood draws occur at baseline (pre-dose) and post-dose at 15 minutes ± 5 minutes, 30 minutes ± 5 minutes, 1 hour ± 10 minutes, 2 hours ± 10 minutes, 4 hours ± 10 minutes, and 6 hours ± 10 minutes.
⁴FMD may be done 7 days prior to the rest of Visit 1 and 5 days prior to the rest of Visit 6.

TABLE 16
Study Drug

| | | |
|---|---|---|
| Study Drug | TV1001 | Placebo |
| Form | Capsule | Capsule |
| Available Unit does | 40 and 80 mg | 40 and 80 mg matched |
| strength(s) | | |
| Route/Administration | Administered orally | Administered orally |
| Supplier | TheraVasc Inc. | TheraVasc Inc. |
| Manufacturer | UPM Pharmaceuticals | UPM Pharmaceuticals |
| | 6200 Seaforth Street, | 6200 Seaforth Street, |
| | Baltimore, MD 21224 | Baltimore, MD 21224 |

Subjects were instructed to return unused study medication and empty packaging at each study visit; all returned capsules were counted and recorded on the appropriate form. Compliance was calculated as the number of capsules taken divided by the number of capsules expected. If a subject was taking fewer capsules than expected, the site staff would counsel the subject on the importance of IP compliance. Investigators were responsible for receipt and proper storage of study medication, as well as for maintaining records of product delivery to site, inventory at site, dispensing of product to each subject, and return of product to TheraVasc, or designee, at the end of the study. All used, unused and partially used medication packages were returned according to TheraVasc, or designee, instructions.

The study was stopped if there were significant changes in safety parameters or significant AEs considered to be related to treatment with study medication (i.e., an imbalance in the safety profile in subjects receiving active drug vs. placebo). An individual subject was withdrawn at the discretion of the responsible investigator and the site study team for the reasons listed below as well as for other safety reasons that may not be listed. In the event one or more subjects were withdrawn, additional subjects were enrolled to ensure an adequate number of subjects complete the cohort. Specific reasons for an individual subject to withdraw included but was not limited to:
Subjects with a pattern of severe adverse events in any SOC, or cardiac monitoring findings as determined by the investigator and/or the sponsor.
Subjects with methemoglobin value ≧15% on any one occasion during study participation.
Subjects with normal baseline blood pressure who experienced any of the following: an increase in blood pressure to 160 mm Hg systolic and/or 90 mmHg diastolic that persists over 24 hours, an increase from baseline blood pressure of 30 mm Hg systolic and/or 15 mm Hg diastolic that persists over 24 hours, any symptomatic increase in blood pressure.
Subjects with stable elevated blood pressure at baseline who experienced any of the following: an increase in blood pressure to 180 mm Hg systolic and/or 100 mmHg diastolic that persists over 24 hours, an increase from baseline blood pressure of 20 mm Hg systolic and/or 10 mm Hg diastolic that persists over 24 hours, any symptomatic increase in blood pressure.
Subjects who experienced a decrease from baseline blood pressure of ≧20 mm Hg systolic with or without an increase of 10 beats per minute (BPM) pulse and the presence of symptoms.

Any subject who developed hypertension or hypotension requiring intervention were followed to resolution, preferably until any intervention therapy was withdrawn.

There were no Data Monitoring Committee (DMC) in place for this study and safety was monitored by the designated Study Medical Expert. A Steering Committee was formed comprising the Sponsor's CEO, two clinicians with experience in clinical trials, a medical regulatory expert and a researcher with expertise in sodium nitrite and its biological affects. CPC provided monthly status reports to the Committee on subject recruitment at each site, monitored reports of the site activities, and other non-safety information regarding the trial. Similar reports were provided in a blinded manner by the distributor of the bottle kits relative to the number of kits distributed to each site, returned bottles, and any issues that arose in randomization or distribution of the IP, assuring that no information was provided to the Committee as to the actual randomization. The Committee would discuss the reports and if any protocol deviations or non-compliance to the investigator's agreement or general investigational plan were noted, action was promptly taken to correct such deviations and secure compliance or discontinue shipments of the investigational drug to the investigator, end the investigator's participation in the investigation, require that all investigational drug be returned to the sponsor, and notified to the FDA. The Committee monitored subject accrual at each site and when necessary discontinue sites that were failing to enroll subjects and add additional sites. The Committee met within two calendar days upon receiving any information that could affect subject safety. The Committee discussed all safety information with CPC, and reported to the FDA and all active clinical investigators any information relevant to the safety of the drug as required under 21 CFR 312.32. The committee made annual reports on the progress of the investigations in accordance with 21 CFR 312.33. No interim analysis was planned for this study.

Study Visits

The study visits included the following components:

### Screening

This visit was conducted within 14 to 21 days of Visit 1—Randomization. A signed informed consent form (ICF) was obtained before any study-specific assessments were performed. The following screening assessments were performed: (1) Informed Consent, (2) Demographics, (3) Medical and Medication History, (4) Physical Exam, (5) Supine Vital Signs, (6) Clinical Safety Labs, (7) Urine Pregnancy Test, (8) Ankle Brachial Index, and (9) Evaluate Inclusion/Exclusion Criteria.

### Visit 1—Randomization

This was considered Day 0 of the study. Subjects were randomized at this visit and given first dose of study medication. The following assessments were performed: (1) Update Medical and Medication History, (2) 12-Lead ECG, (3) Urine Pregnancy Test, (4) FMD (may be performed within 7 days prior to Visit 1), (5) Quality of Life Questionnaires (WIQ and RAND 36), (6) Six-Minute Walk Test, (7) Evaluate Inclusion/Exclusion Criteria, (8) Study Medication Dispensed (the dose of study medication occurred in clinic. Subjects remained on clinic site for safety follow-up until the last PK sampling was complete), (8) PK Sampling (pre-dose and post-dose: 15, 30 minutes ±5 minutes, and 1, 2, 4, 6 hours ±10 minutes), (9) MetHb Sampling (pre-dose and post-dose: 15, 30 minutes ±5 minutes, and 1, 2, 4, 6 hours ±10 minutes), (10) PD Biomarkers, (11) Postural Vital Signs, (12) Adverse Event/Concomitant Medication Assessment (adverse events were captured following administration of the first dose), and (13) Evaluate Study Stopping Criteria.

### Visit 2 (Day 1)

This visit was conducted 1 day (24 hours)+/−4 hours following the time of first dose administration at Visit 1. The subject must have taken the morning dose of the study medication in clinic 30 minutes (+/−10 min) before PK sampling. The following assessments were performed: (1) Administration of morning dose of study medication, (2) Clinical Safety Labs, (3) PK Sampling, (4) MetHb Sampling, (4) Postural Vital Signs, (5) Adverse Event/Concomitant Medication Assessment, and (6) Evaluate Study Stopping Criteria

### Visit 3 (Day 4)

This visit was conducted 4+/−1 days following Visit 1. The subject must have taken the morning dose of the study medication in clinic 30 minutes (+/−10 min) before PK sampling. The following assessments were performed: (1) Administration of morning dose of study medication, (2) Clinical Safety Labs, (3) PK Sampling, (4) MetHb Sampling, (5) Postural Vital Signs, (6) Adverse Event/Concomitant Medication Assessment, (7) Evaluate Study Stopping Criteria (if the subject does not meet stopping criteria but does experience an increase in MetHb to 8% or higher, and optional safety visit at Day 7 was scheduled as described below).

### Optional Safety Visit (Day 7)

This visit was conducted only if the subject had a MetHb at Visit 3 of 8% or higher. It should be conducted 7 days following Visit 1+/−1 day. The subject must have taken the morning dose of the study medication in clinic 30 minutes (+/−10 min) before MetHb sampling. The following assessments were performed: (1) Administration of morning dose of study medication, (2) MetHb Sampling, (3) Postural Vital Signs, (4) Adverse Event/Concomitant Medication Assessment, and (5) Evaluate Study Stopping Criteria

### Visit 4 (Day 14)

This visit was conducted 14+/−2 days following Visit 1. The subject must have taken the morning dose of the study medication in clinic 30 minutes (+/−10 min) before PK sampling. The following assessments were performed: (1) Administration of morning dose of study medication, (2) Clinical Safety Labs, (3) PK Sampling, (4) MetHb Sampling, (5) Urine Pregnancy Test, (6) Postural Vital Signs, (7) Adverse Event/Concomitant Medication Assessment, (8) Evaluate Study Stopping Criteria, (9) Study Medication Compliance, and (10) Study Medication Dispensed

### Visit 5 (Day 28)

This visit was conducted 28+/−2 days following Visit 1. The subject must have taken the morning dose of the study medication in clinic 30 minutes (+/−10 min) before PK sampling. The following assessments were performed: (1) Administration of morning dose of study medication, (2) Clinical Safety Labs, (3) PK Sampling, (4) MetHb Sampling, (5) Postural Vital Signs, (6) 12-Lead ECG, (7) Adverse Event/Concomitant Medication Assessment, (8) Evaluate Study Stopping Criteria, (9) Study Medication Compliance, and (10) Study Medication Dispensed

### Phone Call 1

A phone call was placed to the subject 42+/−2 days following Visit 1. The subject was questioned regarding any adverse events and changes to concomitant medications.

### Phone Call 2

A phone call was placed to the subject 56+/−2 days following Visit 1. The subject was questioned regarding any adverse events and changes to concomitant medications.

### Visit 6 (Day 70)

This visit was conducted 70+/−2 days following Visit 1. The subject must have taken the morning dose of the study medication in clinic 30 minutes (+/−10 min) before PK sampling. The following assessments were performed: (1) Administration of morning dose of study medication (2) Clinical Safety Labs, (3) PK Sampling, (4) MetHb Sampling, (5) PD Biomarkers, (5) Postural Vital Signs, (6) FMD (may be performed within 5 days prior to Visit 6), (7) Quality of Life Questionnaires (WIQ and RAND 36), (8) Six-Minute Walk Test, (9) Adverse Event/Concomitant Medication Assessment, (10) Evaluate Study Stopping Criteria, and (11) Study Medication Compliance

### Visit 7 (Day 71)

This visit was conducted 1 day+1 day following Visit 6. The subject must have taken the morning dose of the study medication (dose escalation) in clinic 30 minutes (+/−10 min) before PK sampling. The following assessments were performed: (1) Study Medication Dispensed, (2) Administration of morning dose of study medication (upon dispensing and administering study medication, subjects were instructed to increase from 1 capsule BID to 2 capsules BID as described. Subject remained in clinic for a 1½ hours post dose observation), (3) Clinical Safety Labs, (4) PK Sampling, (5) MetHb Sampling (subject remained in clinic until results were available), (6) Postural Vital Signs, (7) Adverse Event/Concomitant Medication Assessment, (8) Evaluate Study Stopping Criteria (if the subject did not meet stopping criteria but experienced an increase in MetHb to 8% or higher, a safety visit at Day 70+2 was scheduled as described below in Optional Safety Visit (Visit 7+1), (9) Safety Assessment (immediately prior to subject departure), (10) Evaluation of MetHb results, and (11) Seated Vitals—Pulse Rate and BP.

### Optional Safety Visit (Visit 7+1)

This visit was conducted only if the subject has a MetHb at Visit 7 of 8% or higher. It was conducted 1+1 day following Visit 7. The subject must have taken the morning dose of the study medication in clinic 30 minutes (+/−10 min) before MetHb sampling. The following assessments were performed: (1) Administration of morning dose of study medication, (2) MetHb Sampling, (3) Postural Vital Signs, (4) Adverse Event/Concomitant Medication Assessment, and (5) Evaluate Study Stopping Criteria.

### Visit 8—Termination (Vist 7+6)

This visit was conducted 6+/−1 days following Visit 7. The subject must have taken the morning dose of the study medication in clinic 30 minutes (+/−10 min) before PK sampling. This would be the final dose and study visit. The following assessments were performed: (1) Physical Exam, (2) Clinical Safety Labs, (3) PK Sampling, (4) MetHb Sampling, (5) Urine Pregnancy Test, (6) Postural Vital Signs, (7) 12-Lead ECG, (8) Adverse Event/Concomitant Medication Assessment, and (9) Study Medication Compliance

### Follow-up Phone Call

A phone call was placed to the subject 7+/−1 days following Visit 8. If this subject early terminates from the study, a phone call was placed to the subject 7 days following the ET visit +/−1 day. The subject was questioned regarding any adverse events and changes to concomitant medications.

### Early Termination Visit (ET)

In the case that a subject must withdraw early from study participation for any reason prior to Visit 6, every effort was made to complete an early termination visit. The subject must have taken the morning dose of the study medication in clinic 30 minutes (+/−10 min) before PK sampling unless the subject was withdrawn for safety and should stop taking IP immediately. The following assessments were performed: (1) Administration of morning dose of study medication, if applicable, (2) Physical Exam, (3) Clinical Safety Labs, (4) PK Sampling, (5) MetHb Sampling, (6) PD Biomarkers, (7) Postural Vital Signs, (8) 12-Lead ECG, (9) FMD (may be performed within 5 days prior to ET Visit), (10) Quality of Life Questionnaires (WIQ and RAND 36), (11) Six-Minute Walk Test, (12) Urine Pregnancy Test, (13) Adverse Event/Concomitant Medication Assessment, and (14) Study Medication Compliance. Moreover, if early termination occurred after Visit 6 but before the appropriate visit window for Visit 8, all procedures required at Visit 8 were completed.

Selection and Withdrawal of Subjects

The inclusion criteria included subjects between the ages of 35 and 85 years. Subjects must be either male or females post-menopausal, sterilized or using suitable birth control. Suitable birth control must be total abstinence, male partner sterilization or double barrier method paired with using oral contraception, injectable progestogen, implants of levonorgestrel, estrogenic vaginal ring, percutaneous contraceptive patches, or intrauterine device (IUD). A history of peripheral artery disease (PAD) was confirmed by medical chart or an ankle brachial index at rest of ≦0.90. If subjects received a medical standard treatment for cardiac risk factors, subject must have been on a stable treatment for at least 1 month prior to Screening. If included in this regimen, treatments such as cilostazol, pentoxifylline, statins, or angiotensin converting enzymes (ACE)-inhibitors; supervised exercise rehabilitation training; participation in a formal smoking cessation program or prescription of medications for smoking cessation were not changed significantly in the last month and were not expected to change over the duration of the study. If subjects experienced claudication symptoms, subjects must have stable lower extremity symptoms for at least 1 month (e.g. no change in claudication symptoms) prior to Screening. Subjects were required to provide written informed consent and willingness as documented by a signed informed consent form.

The exclusion criteria included subjects with non-atherosclerotic PAD (e.g. Buerger's vasculitis), lower extremity surgical or percutaneous revascularization, evidence of graft failure or other peripheral vascular surgical procedure within the last 6 months prior to Screening, anticipated lower extremity revascularization within the treatment period, myocardial infarction, unstable angina, cerebrovascular accident or transient ischemic attack (TIA) within 3 months prior to Screening, poorly controlled diabetes (HgAlc>10.0), poorly controlled hypertension (systolic blood pressure (SBP)≦160 mmHg or diastolic blood pressure (DBP)≦100 mmHg) despite therapy, systolic blood pressure ≦100 mmHg on current medical regimen, hypersensitivity to sodium nitrite or related compounds, and renal insufficiency documented as eGFR <30 mL/minute/1.73 m² (Modification of Diet in Renal Disease Study MDRD). Exclusion criteria also included subjects who were pregnant or nursing women, who had a life expectancy of <6 months, a chronic illness that may increase the risks associated with this study in the opinion of the investigator, an active malignancy requiring active anti-neoplastic therapy that, in the opinion of the investigator, interfered with study treatment or participation (although stable basal cell skin cancer was allowed and cancer being treated solely with hormonal therapy was allowed), an active infection (i.e. systemic or osteomyelitis), a NYHA CHF Class III or IV, has had recent hospitalization (<30 days) for acute coronary syndrome (ACS), myocardial infarction (MI), congestive heart failure (CHF) or stroke, recent (<30 days) coronary revascularization had previously been treated with angiogenic factors or stem cell therapy within 1 year prior to Screening, was involved in another PAD clinical trial within past 1 month prior to Screening, had exposed tendon, muscle or bone or a diagnosis of critical leg ischemia (CLI), was previously amputated within 3 months prior to Screening, or had a planned amputation that would limit walking (although small toe is allowed). Exclusion criteria also included subjects whose ability to perform the 6 minute walk test was limited by symptoms other than claudication, who was diagnosed with alcohol or other substance abuse, had a history of methemoglobinema, (metHb ≧15%), who had an inability to speak English (due to need for administering standardized English-language questionnaire), who had evidence of anemia or a history of chronic hemolytic condition, including sickle cell disease, who had chronic use of anti-migraine medication such as Imitrex or sumatriptan, and a positive screen for glucose-6-phosphate dehydrogenase (G6PD) deficiency at screening. Subjects who chronically took the following medications: Allopurinol, PDE-5 inhibitors, sedative tricyclic antidepressants, sedative antihistamines, meperidine and related narcotic central nervous system (CNS) depressants, and nitrates were also excluded.

The withdrawal criteria allowed a subject to withdraw from the study at any time at his/her own request. The subject may also have been withdrawn at the Investigator's request if it was the Investigator's opinion that it was not in the subject's best interest to continue in the study. The subject was withdrawn if he or she met stopping criteria described above. In the event a subject was withdrawn from the study for any reason, the subject was followed-up with reasonable effort made to determine the reason for their withdrawal from the study and an ET visit as described above. Telephone calls, certified letters and offers of transportation assistance were considered reasonable effort. A summary of subject withdrawals is provided in Table 17.

TABLE 17
Subject Withdrawals
SUMMARY OF SUBJECT DISPOSITION

| | Placebo | 40 mg | 80 mg |
|---|---|---|---|
| | n = 18 | n = 19 | n = 18 |
| Subjects who completed the | 15 (83.3%) | 17 (89.5%) | 15 (83.3%) |
| study | | | |
| Subjects who withdrew prior | 3 (16.7%) | 2 (10.5%) | 3 (16.7%) |
| to completion | | | |
| Reasons for withdrawal: | | | |
| Adverse Event | 0 (0.0%) | 1 (5.3%) | 2 (11.1%) |
| Met withdrawal-new | 1 (5.6%) | 0 (0.0%) | 1 (5.6%) |
| hypotension | | | |
| Subject Request-lack of | 0 (0.0%) | 1 (5.3%) | 0 (0.0%) |
| energy | | | |
| Subject request-refused to | 1 (5.6%) | 0 (0.0%) | 0 (0.0%) |
| continue | | | |
| Subject request-no benefit | 1 (5.6%) | 0 (0.0%) | 0 (0.0%) |

Treatment of Subjects

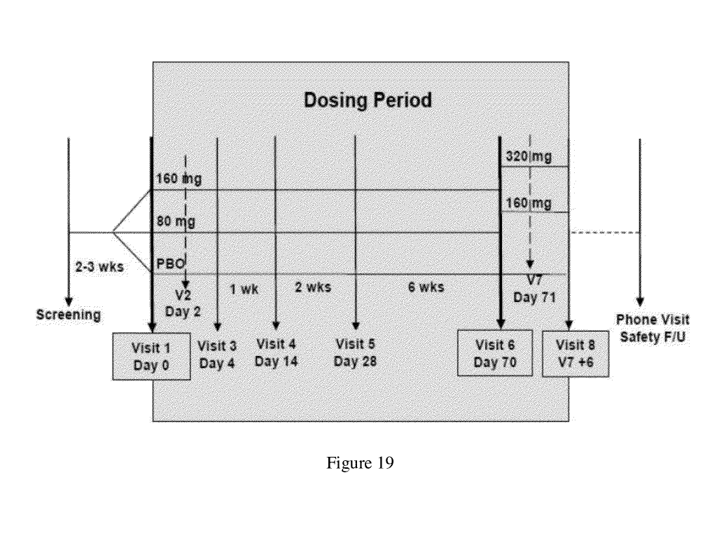

The three dosing arms are placebo, 40 mg BID and 80 mg BID sodium nitrite as shown in FIG. 19. All doses were given as a twice-daily oral dose for 10 weeks. On the day following the 10 week dosing period and completion of efficacy assessments, subjects in each treatment arm entered a 6 day dose-escalation period (dose-doubling). Subjects in the 40 mg sodium nitrite BID group were dose-escalated to 80 mg sodium nitrite BID for 6-days, and subjects in the 80 mg sodium nitrite BID were dose-escalated to 160 mg sodium nitrite BID for 6-days. Placebo subjects doubled the number of placebo capsules taken BID. All study medication was stopped at the end of the 6-day dose-escalation period.

Subjects chronically taking Imitrex (sumatriptan), allopurinol, PDE-5 inhibitors, sedative tricyclic antidepressants, sedative antihistamines, meperidine and related narcotic CNS depressants, and nitrates were prohibited from participating in this study.

Subjects were instructed to return unused study medication at each study visit; all returned capsules were counted and recorded on the appropriate form. Compliance was calculated as the number of capsules taken divided by the number of capsules expected. The number of capsules taken was calculated by subtracting the number of capsules left from 50, the number of capsules in the bottle. If a subject took fewer capsules than expected, the site staff counseled the subject on the importance of IP compliance. Investigators were responsible for receipt and proper storage of study medication, as well as for maintaining records of product delivery to site, inventory at site, dispensing of product to each subject, and return of product to TheraVasc, or designee, at the end of the study. All used, unused and partially used medication packages were returned according to TheraVasc, or designee, instructions.

Assessment of Efficacy

The efficacy parameters included: (1) flow-mediated vasodilation (FMD), six-minute walk test, pharmacokinetics (PK), biomarkers/pharmacodynamic (PD) markers, and quality of life questionnaires (QoL),

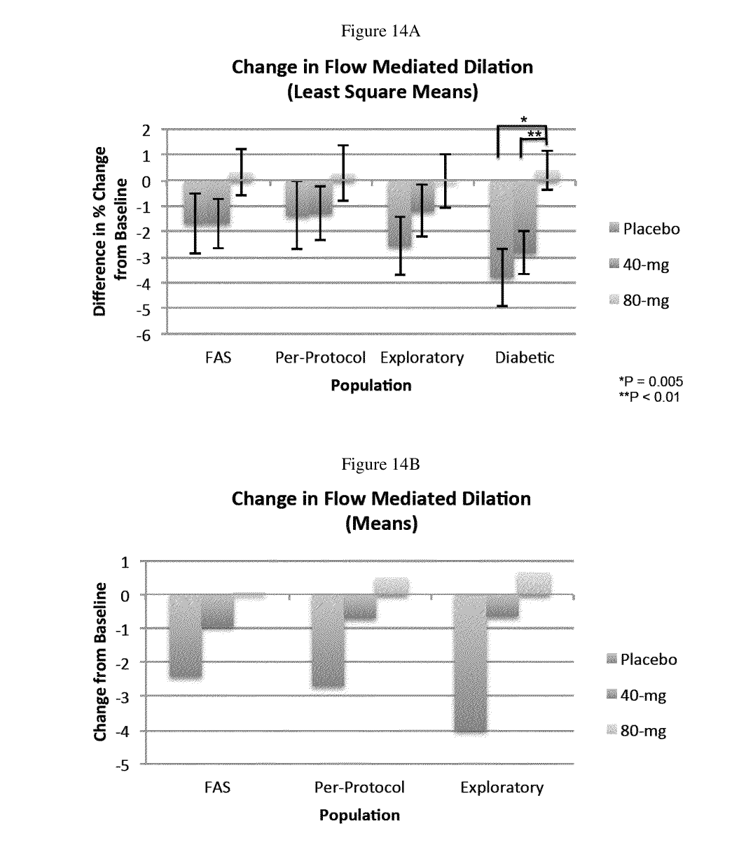

FMD was assessed using brachial arterial reactivity (BAR). FMD assessed the percent change of flow-mediated vasodilation of the brachial artery. The FMD was completed fasting and prior to the six-minute walk test. In the event that an individual site does not have the capability to complete the FMD on-site, the FMD was performed up to 7 days prior to the Visit 1 and up to 5 days prior to Visit 6 at an approved central site which also participated in the study and was within a reasonable distance to travel. Either the PI or a sub-Investigator with appropriate medical training assessed the quality of the FMD image before proceeding with either the initial dose or increased dose of IP after Visit 6. The data obtained during the FMD procedure was submitted to Vas Core FMD core lab and detailed in FIGS. 14A-B. Overall, positive trends were observed and particular in the diabetic population, significant (p<0.005) improvement in FMD was seen with 80 mg dose.

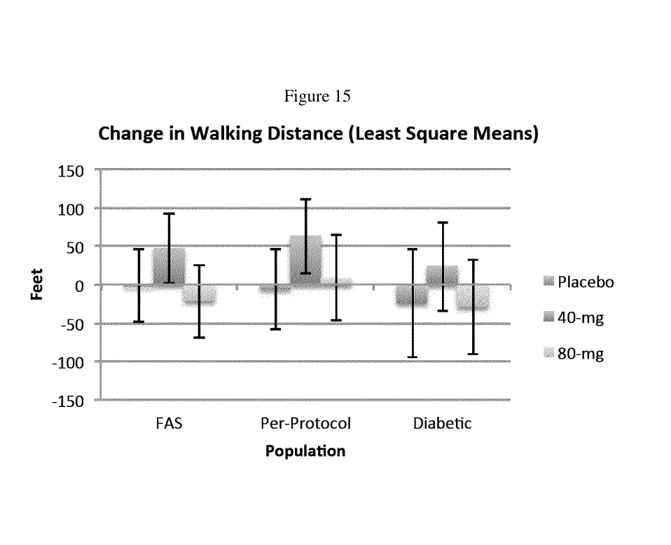

A six-minute walk was performed to assess the distance a subject walked over a 6 minute period. The course length was either 50 or 100 feet dependent upon the set up of the research facility. The subject walked at a self-directed pace for 6 minutes. Results are shown in FIG. 15. Overall, a strong improvement was seen in the 40 mg group.

Blood sample for analysis of plasma concentration of sodium nitrite was collected at the time points listed in Table 15—Schedule of Assessments. Other than V1, PK sampling occurred 30 minutes (+/−10 min) after subject took the morning dose of study medication in the clinic.

On V1, PD sampling occurred prior to subject receiving study medication. On V6, the subject took the morning dose of study medication in clinic at 30 minutes (+/−10 min) before PD sampling. Blood samples for analysis of biomarkers (e.g., sodium nitrite, nitrite, nitrate, SICAM, VCAM, F2-isoprostanes and IL-6) were collected at the time points listed in Table 15-Schedule of Assessments. Other biomarkers of inflammation and metabolism may also be explored.

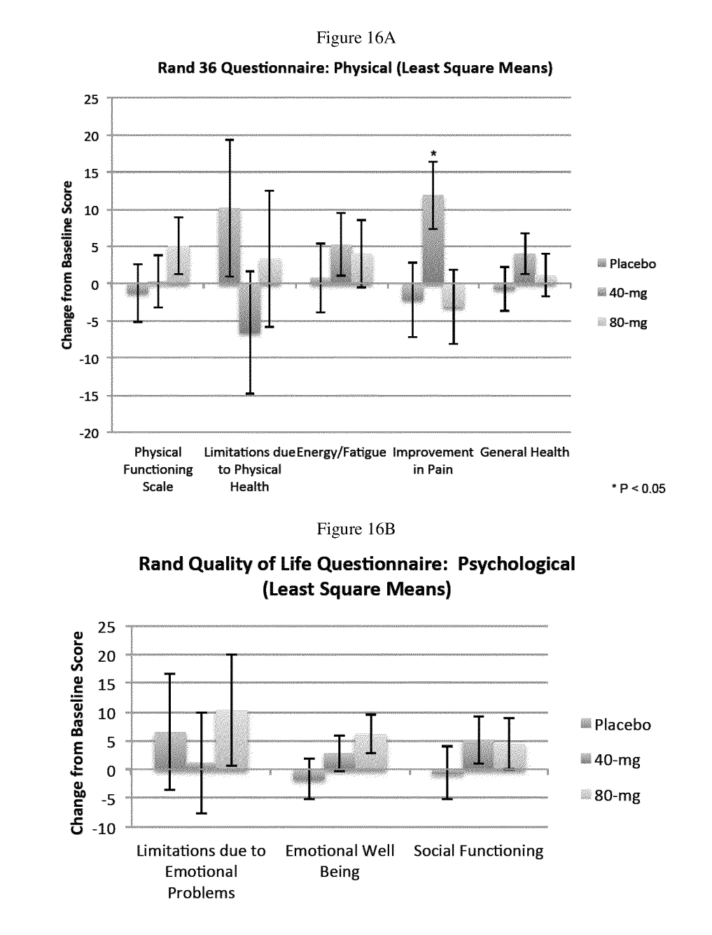

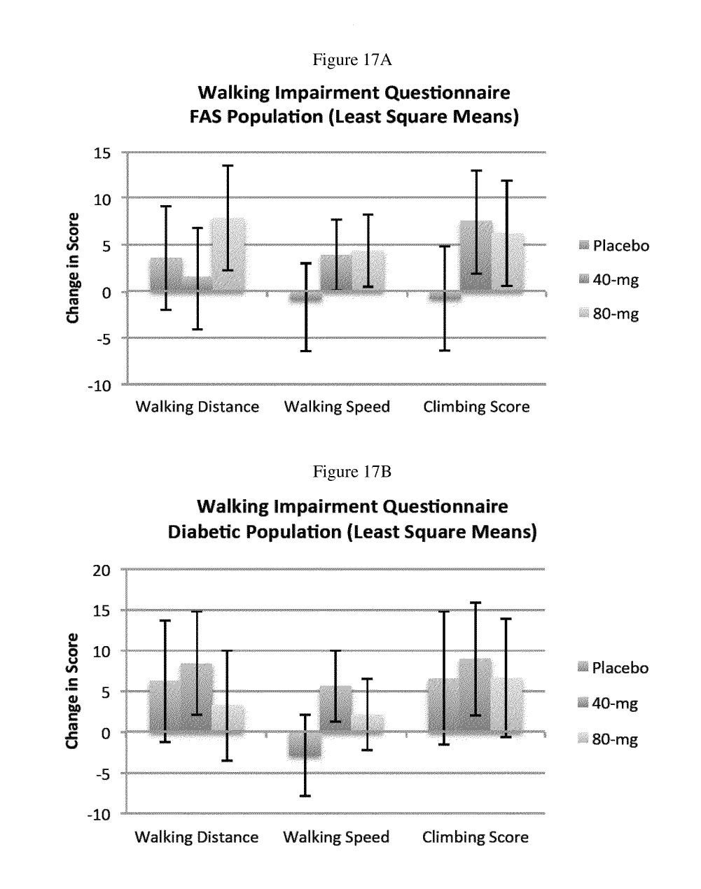

Quality of life was measured by two questionnaires: WIQ and RAND 36. The two questionnaires were administered in the same sequence: WIQ first, followed by the RAND 36. The WIQ was a disease-specific instrument that measures community-based walking. The questionnaire consisted of four subscales (pain severity, distance, speed, and stairs). The WIQ was verbally administered to the subject by the Investigator, or designee. The RAND 36 was an instrument that measured general health issues. Study staff directed subjects to complete the RAND 36 on their own. Staff did not try to interpret the questions for the subject. If the subject did not understand a particular question, the study staff instructed the subject to interpret the meaning of the question to the best of his or her ability and provide an answer that seems most accurate to the subject. No family members or other individuals were allowed to answer questions or complete the questionnaire for the subject. All questionnaires were completed directly on the written source document pages. The study coordinator reviewed all questionnaires to ensure that there was only one response to each question, each question has been answered and any necessary corrections have been initialed and dated by the Investigator (or designee) or subject, accordingly. The results from the RAND 36 physical and psychological assessments are detailed in FIGS. 16A-B. RAND 36 showed a trend toward improvement in quality of life assessment and significant improvement in pain assessment in the 40 mg group. Results from the WIQ assessments are detailed in FIGS. 17A-B. WIQ showed no change in assessment in walking distance and a trend toward improvement in walking speed and stair climbing.

Assessment of Safety

The following safety parameters were assessed: medical and medication history, concomitant medication usage, physical examination, vital signs, 12-lead ECG, clinical chemistries, CBC, urinalysis, and adverse events. Urine pregnancy testing was completed for women of child-bearing potential who have not been surgically sterilized. Assessment of acute adverse events (i.e., drop in blood pressure, dizziness) after administration of 1^{st} dose for each dose level of sodium nitrite. Dose-limiting toxicity (DLT) was defined as Grade 3 and clinically significant hematological events, particularly MetHb.

Overall, no severe adverse effects were observed in treated groups. Dose dependent hypotensive affects were observed demonstrating the hemodynamic affects of the treatment. Moreover, Methemoglobin levels were of no concern, even at the 160 mg dose escalation.

Demographic information (Table 18) and a complete medical history (Table 19) were obtained at the Screening Visit. Medical history for any ongoing ailments and for 5 years prior to screening and medication history for the past 1 month were documented. Medical and medication history was reviewed with the subject prior to randomization to ensure all data were accurate and complete to date.

TABLE 18
Demographic Data

| | | Placebo | 40-mg | 80-mg |
|---|---|---|---|---|
| | | n = 18 | n = 19 | n = 18 |
| Age at informed consent (years) | | 64.9 +/− 8.98 | 65.3 +/− 8.86 | 67.9 +/− 9.99 |
| Gender | Male | 13 (72.2%) | 15 (78.9%) | 13 (72.2%) |
| | Female | 5 (27.8%) | 4 (21.1%) | 5 (27.7%) |

| | | | |
|---|---|---|---|
| Race/Ethnicity | | | |
| Black or African American | 5 (27.8%) | 6 (31.6%) | 8 (44.4%) |
| White | 12 (66.7%) | 12 (63.2%) | 10 (55.6%) |
| Other | 1 (5.6%) | 1 (5.3%) | 0 (0.0%) |
| Weight (kg) | 88.07 +/− 27.24 | 79.32 +/− 13.53 | 88.99 +/− 16.70 |
| Height (cm) | 173.18 +/− 13.29 | 172.01 +/− 9.87 | 172.18 +/− 9.95 |
| Screening BMI (kg/m2) | 29.32 +/− 8.31 | 26.71 +/− 2.99 | 30.01 +/− 5.03 |
| ABI in index limb at screening | 0.56 +/− 0.15 | 0.62 +/− 0.20 | 0.69 +/− 0.17 |
| Diabetes Diagnosis | 10 (55.6%) | 14 (73.7%) | 14 (77.8%) |
| Hb A1c (% Hb) at screening | 6.97 +/− 1.48 | 6.99 +/− 1.27 | 6.71 +/− 0.94 |

TABLE 19
Medical History Background

| | Placebo | 40-mg | 80-mg |
|---|---|---|---|
| | N = 18 | N = 19 | N = 18 |
| PAD in last 5 years | 18 (100%) | 19 (100%) | 18 (100%) |
| Peripheral revascularization | 8 (44.4%) | 2 (10.5%) | 8 (44.4%) |
| in last 5 years | | | |
| Coronary artery disease | 6 (33.3%) | 5 (26.3%) | 7 (38.9%) |
| in last 5 years | | | |
| Angina | 2 (11.1%) | 0 | 4 (22.2%) |
| Myocardial infarction | 0 | 2 (10.5%) | 2 (11.1%) |
| Coronary revascularization | 1 (5.6%) | 0 | 4 (22.2%) |
| in last 5 years | | | |
| Congestive Heart Failure | 1 (5.6%) | 0 | 1 (5.6%) |
| Cerebrovascular disease | 2 (11.1%) | 3 (15.8%) | 5 (27.8%) |
| in last 5 years | | | |
| Ischemic stroke | 0 | 1 (5.3%) | 1 (5.6%) |
| TIA.mini-stroke | 1 (5.6%) | 0 | 1 (5.6%) |
| Hypertension | 16 (88.9%) | 18 (94.7%) | 16 (88.9%) |
| Dyslipidemia | 15 (83.3%) | 18 (94.7%) | 16 (88.9%) |
| Diabetes type 1 | 0 | 1 (5.3%) | 0 |
| Diabetes type 2 | 10 (55.6%) | 12 (63.2%) | 12 (66.7%) |
| Deep vein thrombosis/ | 0 | 0 | 2 (11.1%) |
| Pulmonary Embolism | | | |
| Stent/Balloon/Bypass | 5 (27.8%) | 0 | 1 (5.6%) |

ABI assessments were measured at the screening visit in order to assess if the subject was appropriate according to inclusion criteria. ABI assessments were done only after the subject had been resting in a supine position for at least 10 minutes. The ABI was defined as the ratio between the higher of the two pedal systolic blood pressures (dorsalis pedis and posterior tibialis) and the higher of the two systolic brachial pressures. A continuous wave Doppler, between 5 and 10 MHz, was used to measure the systolic pressures in both the dorsalis pedis and posterior tibial arteries in each leg, as well as the brachial arteries in each arm. The higher of the 2 arm pressures and the higher of the 2 ankle pressures for each leg were used for the calculation. The ABI was calculated for both legs. The ABI must be less than 0.90 in at least one extremity to qualify for the study.

Site staff recorded any medication taken by a subject after randomization into the study, including prescribed, nutritional supplements and over-the-counter medications, and the reason for its use as a concomitant medication. If a subject required treatment by any medications listed as a prohibited concomitant therapy, he or she would be withdrawn from study participation and completed an ET visit.

A complete physical examination was performed at Screening and included height, weight and assessments of the following systems: general appearance; eyes; ears, nose, and throat; head and neck; chest and lungs; cardiovascular; abdomen; musculoskeletal; lymphatic; dermatological; neurological; and extremities. At Visit 8 or Early Termination a follow-up physical exam assessed weight and any changes to the above mentioned systems. Any significant changes noted at Visit 8 were documented as an adverse event unless otherwise noted by the PI or designee.

Supine vital signs were measured at the Screening Visit. The subject rested in a supine position for a minimum of 3 minutes prior to obtaining vital sign measurements. Vital signs included BP and pulse rate. Postural vital signs, including both supine and standing measurements of blood pressure and pulse rate, were recorded at all study visits following the first dose of IP administration. Measurements were performed as follows: (1) the subject rested in a supine position for a minimum of 3 minutes, (2) vital signs (BP and pulse rate) were measured while the subject was supine, (3) the subject assumed a standing position for a minimum of 5 minutes, and (4) vital signs (BP and pulse rate) were measured while the subject was standing. Pulse rate and blood pressure data are detailed in Table 20.

TABLE 20
Pulse Rate and Blood Pressure

| | | Screening | Visit 1 | Visit 2 | Visit 3 | Visit 4 | Visit 5 | Visit 6 | Visit 7 | Visit 8 |
|---|---|---|---|---|---|---|---|---|---|---|
| Supine (Mean) | | | | | | | | | | |
| Pulse | Placebo | 73.6 | 74.4 | 73.0 | 71.0 | 74.8 | 74.6 | 73.1 | 73.1 | 75.6 |
| | 40-mg | 71.4 | 74.1 | 74.1 | 74.7 | 71.7 | 72.7 | 70.4 | 73.1 | 70.2 |
| | 80-mg | 63.9 | 65.6 | 65.1 | 66.7 | 64.6 | 68.7 | 65.5 | 64.3 | 68.3 |
| Blood Pressure | Placebo | 141.3/77.9 | 141.4/78.3 | 139.9/78.4 | 138.4/77.4 | 137.8/75.4 | 140.3/76.9 | 145.4/79.5 | 139.8/77.8 | 136.1/75.1 |
| | 40-mg | 136.8/75.8 | 129.7/72.3 | 128.0/70.5 | 129.8/72.5 | 128.4/71.1 | 124.1/72.0 | 127.3/73.7 | 126.7/71.3 | 130.0/72.2 |
| | 80-mg | 132.4/69.4 | 129.8/68.4 | 122.8/66.7 | 127.1/66.7 | 125.1/65.2 | 124.6/68.9 | 123.1/66.2 | 118.4/64.4 | 120.7/66.9 |
| Standing (Mean) | | | | | | | | | | |
| Pulse | Placebo | | 78.1 | 77.8 | 74.9 | 78.1 | 78.5 | 76.0 | 76.1 | 77.4 |
| | 40-mg | | 75.8 | 78.6 | 76.7 | 75.6 | 76.6 | 73.9 | 76.3 | 74.3 |
| | 80-mg | | 72.6 | 72.3 | 72.9 | 72.6 | 72.5 | 67.6 | 70.4 | 72.6 |
| Blood Pressure | Placebo | | 141.6/81.7 | 144.3/81.2 | 139.9/79.4 | 139.9/80.3 | 137.7/79.4 | 143.3/78.4 | 141.3/78.7 | 138.2/75.0 |
| | 40-mg | | 129.5/73.1 | 128.3/72.6 | 129.4/73.1 | 124.9/71.0 | 124.3/73.6 | 127.6/73.2 | 122.2/73.2 | 123.1/68.7 |
| | 80-mg | | 125.4/70.2 | 123.1/71.9 | 124.8/69.2 | 123.7/68.8 | 119.5/73.9 | 124.8/70.0 | 123.3/67.1 | 117.7/67.9 |
| Orthostatic Changes | | | | | | | | | | |
| Pulse | Placebo | | 3.7 | 4.8 | 3.9 | 3.3 | 3.9 | 2.9 | 3.0 | 1.8 |
| | 40-mg | | 1.8 | 4.5 | 2.0 | 3.9 | 3.8 | 3.5 | 3.2 | 4.1 |
| | 80-mg | | 6.9 | 7.3 | 6.2 | 7.9 | 3.8 | 2.1 | 6.1 | 4.3 |
| Systolic BP | Placebo | | 0.2 | 4.3 | 1.6 | 2.1 | −2.6 | −2.1 | 1.5 | 2.2 |
| | 40-mg | | −0.3 | 0.3 | −0.4 | −3.4 | 0.3 | 0.3 | −4.5 | −6.9 |
| | 80-mg | | −4.4 | 0.3 | −2.3 | −1.4 | −5.2 | 1.6 | 4.8 | −3.0 |
| Diastolic BP | Placebo | | 3.3 | 2.8 | 1.9 | 4.9 | 2.5 | −1.1 | 0.9 | −0.1 |
| | 40-mg | | 0.8 | 2.1 | 0.6 | −0.1 | −0.4 | −0.5 | 1.9 | −3.5 |
| | 80-mg | | 1.8 | 5.2 | 2.5 | 3.5 | 3.0 | 3.8 | 2.7 | 1.0 |

A resting 12-lead ECG printout with the subject in supine position was obtained at the time points listed in Table 15-Schedule of Assessments. All ECGs were assessed by the PI or qualified designee for clinical significance of any abnormalities or changes and documented on the ECG source document. Any clinically significant abnormalities that occurred after the first dose of sodium nitrite were recorded as AEs on the eCRF. The 12-lead ECG was obtained immediately following vitals, with the exception of the Visit 1 Randomization day ECG which was collected prior to dosing. The ECG data details are provided in Table 21.

TABLE 21
ECG

| | Visit 1 | Visit 5 | Visit 8 |
|---|---|---|---|
| Heart Rate | | | |
| (beats/minute) | | | |
| Placebo | 72.1 +/− 13.9 | 71.7 +/− 15.1 | 73.0 +/− 12.2 |
| 40-mg | 71.4 +/− 12.7 | 72.2 +/− 14.8 | |
| 80-mg | 62.7 +/− 10.7 | 65.5 +/− 11.9 | 74.3 +/− 16.9 |
| 160-mg | | | 64.7 +/− 10.0 |
| QTcB interval (msec) | | | |
| Placebo | 433.2 +/− 33.0 | 430.9 +/− 24.0 | 438.6 +/− 35.3 |
| 40-mg | 415.9 +/− 49.0 | 430.1 +/− 34.8 | |
| 80-mg | 422.3 +/− 34.0 | 411.6 +/− 49.7 | 423.2 +/− 40.3 |
| 160-mg | | | 427.7 +/− 31.9 |
| QTcF interval (msec) | | | |
| Placebo | 421.2 +/− 31.4 | 419.7 +/− 22.5 | 425.4 +/− 33.9 |
| 40-mg | 404.8 +/− 44.9 | 417.7 +/− 24.0 | |
| 80-mg | 419.9 +/− 30.5 | 406.2 +/− 46.2 | 409.5 +/− 34.3 |
| 160-mg | | | 422.8 +/− 27.9 |

QTc changes >60 msec: serious;
QTc changes >30 msec: questionable

Laboratory evaluations were collected at the time points listed in Table 15-. All safety clinical laboratory testing was performed by a central laboratory, with the exception of the urine pregnancy test and methemoglobin which was completed on-site. Specimen samples were sent from the investigative site to the central laboratory. A urine pregnancy test was performed at the time points listed in Table 15 if any woman was not surgically sterilized or post-menopausal.

Clinical Labs were performed with subjects fasting and include the following: Urinalysis: Protein dipstick, specific gravity, appearance, pH, glucose, blood, bilirubin, ketones, and microscopic examination. Clinical chemistry panel included: albumin, alkaline phosphatase, serum amylase, ALT, AST, BUN, calcium (serum), serum chloride, CO2, serum creatinine, direct bilirubin, Gamma-GT, glucose, LDH, serum phosphorus, potassium, sodium, total bilirubin, total protein, uric acid, total cholesterol, LDL, HDL, triglycerides, and HbA1c (Screening only). Hematology panel included: WBC, RBC, Hb, Hct, MCV, MCH, MCHC, Platelets, and RDW.

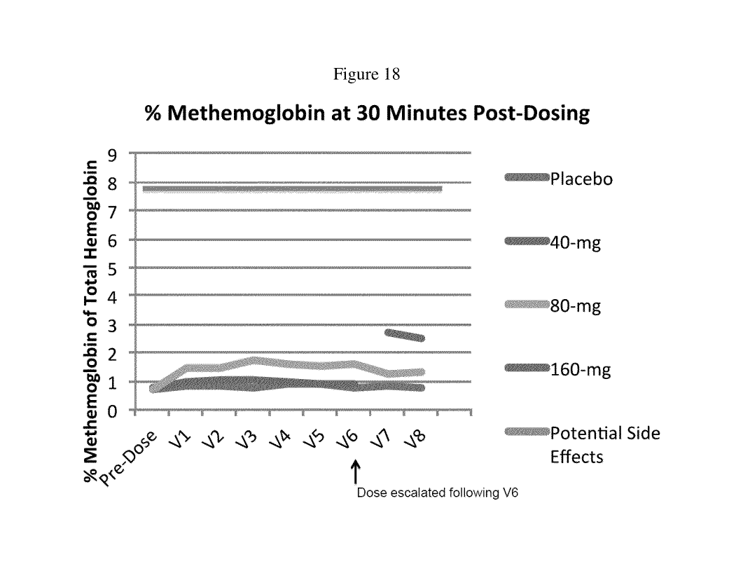

Methemoglobin was measured locally for safety along with each Clinical Lab with the following exceptions: Methemoglobin was not collected at screening, Methemoglobin was not collected serially at 7 time points at V1, and Methemoglobin was not collected alone at the “Optional Safety Visit,” if applicable. FIG. 18 is a graph showing the % Methemoglobin at 30 minutes post-dosing for V1-V8.

Female subjects in this study who were not post-menopausal or sterilized were required to be using of the following methods of birth control: total abstinence defined as sexual inactivity which is consistent with the preferred and usual lifestyle of the subject, periodic abstinence (e.g., calendar, ovulation, symptothermal, post-ovulation methods) and withdrawal were not acceptable, male partner sterilization prior to the female subject's entry into the study; and this male was the sole partner for the subject, double barrier method defined as condom and occlusive cap (diaphragm or cervical/vault caps) plus spermicidal agent (foam/gel/film/cream/suppository) combined with pharmaceutical contraception listed below:
Oral contraception, either combined or progestogen alone
Injectable progestogen
Implants of levonorgestrel
Estrogenic vaginal ring
Percutaneous contraceptive patches
Intrauterine device (IUD) or intrauterine system (IUS) that meets the <1% failure rate as stated on the product label

Any subject who becomes pregnant during the study was not eligible to continue in the study and completed end of study procedures at that time. Male subjects and their partners were expected to use appropriate birth control methods or abstain from sexual intercourse. Male subjects agreed to inform the Investigator immediately if their partner becomes pregnant during the course of the study monitoring period.

Complete pregnancy information, including the outcome of the pregnancy, was collected in the source documents on any female subject or partner of a male subject (if she was willing) who became pregnant during this study monitoring period. In the absence of complications, follow-up was no longer than 6 to 8 weeks following the delivery date. Any premature terminations, whether elective, therapeutic, or spontaneous, were reported. While pregnancy itself was not considered to be an adverse effect, any pregnancy complications, including a spontaneous termination or elective termination for medical reasons, should be reported as an adverse effect. A spontaneous abortion was considered to be an SAE. Any SAE occurring as a result of a post-study pregnancy and considered reasonably related to the investigational product by the Investigator was reported to the Sponsor.

As defined by the International Conference on Harmonisation (ICH), an AE was any untoward medical occurrence in a patient or clinical investigation subject administered an investigational product, whether or not the event was considered related to the investigational product. An AE was therefore any unfavorable and unintended sign (including an abnormal laboratory finding), symptom, or disease (new or exacerbated) temporally associated with the use of the investigational product and was collected starting when IP was administered. Examples of an AE include conditions newly detected or diagnosed after investigational product administration, including conditions that may have been present but undetected prior to the start of the study, conditions known to have been present prior to the start of the study which worsen after the administration of the investigational product, signs, symptoms, or the clinical sequelae of a suspected drug interaction, and signs, symptoms, or the clinical sequelae of a suspected overdose of either investigational product or a concurrent medication (overdose per se was not reported as an AE). Examples of issues not considered an AE include: medical or surgical procedures (e.g., endoscopy, appendectomy); a condition that leads to a procedure is an AE if it qualifies according to the definitions above, situations where an untoward medical occurrence has not occur (e.g., social, observational, diagnostic, or convenience admission to a hospital), fluctuations of pre-existing disease(s) or condition(s) present or detected at the start of the study that do not represent a clinically significant exacerbation, and abnormal laboratory or test findings that were not assessed by the PI or a sub-Investigator with appropriate medical training as clinically significant. A summary of adverse events is detailed in Table 22.

TABLE 22
Summary of Adverse Events

| | Placebo | 40 mg | 80 mg |
|---|---|---|---|
| Overall: | | | |
| Number (%) of Subjects with | 9 (50.0%) | 12 (63.2%) | 14 (77.8%) |
| at least one AE | | | |
| Number (%) of Subjects with | 9 (50.0%) | 12 (63.2%) | 14 (77.8%) |
| at least one TEAE | | | |
| Number of AEs | 15 | 32 | 40 |
| Number of TEAEs | 15 | 32 | 39 |
| Number of SAEs | 2 | 0 | 0 |
| Number of TEAEs by Severity | | | |
| Mild | 12 | 26 | 31 |
| Moderate | 3 | 6 | 8 |
| Number of TEAEs by | | | |
| Relationship to Study Drug | | | |
| Not Related | 12 | 10 | 9 |
| Possibly Related | 3 | 22 | 24 |
| Probably Related | 0 | 0 | 6 |
| Six-Day Dose-Escalation | | | |
| Period Only: | | | |
| Number (%) of Subjects with | 2 (11.1%) | 3 (15.8%) | 7 (38.9%) |
| at least on TEAE | | | |
| Number of TEAEs | 2 | 3 | 11 |

Statistical Methods

Demographic data, clinical chemistry, CBCs, biomarkers, and adverse events were summarized in tabular form by dose level and overall. Descriptive statistics were used to summarize the demographic and clinical data, such as ECGs and vitals. Laboratory values above and below the normal limit were flagged, and adverse events presented by SOC, severity and relationship to study treatment.

The primary efficacy analysis was compare to the change from baseline and Day 70 (Visit 6) of FMD between the pooled-drug and placebo treated groups following 10 weeks of treatment using an unpaired t-test. In case of a substantially skewed distribution within the comparison groups, a nonparametric two sample Wilcoxon signed-rank test was used. For dichotomized efficacy endpoints the null hypothesis H0: rc=rp versus H1: rc≠p was tested, where rc is the proportion of subjects with improved results in BID cohort and rp was the proportion of subjects with improved results in the placebo cohort. The differences between groups were tested with chi square test or Fisher exact test. Secondary analyses employed repeated measures ANOVA based on Generalized Estimating Equations to incorporate time, group and interaction. Other confounding variables were included in the baseline covariates framework. Analysis of the secondary endpoints such as 6-minute walk and QoL questionnaires was performed as described for the primary efficacy analysis. All statistical decisions were made before un-blinding.

Additionally, plasma levels of sodium nitrite were tabulated and plotted as a log-dose response curve. Functional parameters were tabulated by dose and overall. Summary statistics were computed and log-dose response curves were prepared for each parameter as appropriate.

A statistical analysis plan was developed to detail the statistical approach, particular contrasts of interest, and additionally include any exploratory or unadjusted analysis of the primary efficacy endpoints by treatment group.

With a total sample size of 50 subjects (n=34 sodium nitrite; n=16 placebo), the study had ˜82% power to detect a difference in the means of sodium nitrite (pooled-groups) compared with placebo for the efficacy endpoint of FMD at the 0.050 two-sided level of significance. Specifically, with approximately 34 subjects in the pooled sodium nitrite group and 16 subjects in the placebo group, the study had 82.19% power to detect a 1.4% difference in FMD responses between sodium nitrite treated subjects compared with placebo treated subjects after 10 weeks of treatment with 1.6% standard deviations (SD). The sample size was thus empirically determined to be sufficient for this early-stage, clinical study. Accounting for drop-outs, a sample size of up to 60 subjects (20 subjects/group) was sufficient to account for drop-outs as needed to achieve a final sample size of approximately 17 subjects per group. Last observation carried forward (LOCF) was applied to missing data.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All references, patents, patent application publications, and patent applications cited herein are hereby incorporated by reference to the same extent as if each of these references, patents, patent application publications, and patent applications were separately incorporated by reference herein.

What is claimed is:

1. A method of treating peripheral artery disease, said method comprising administering to a subject in need thereof a pharmaceutical composition comprising an effective amount of inorganic nitrite, or a pharmaceutically acceptable salt thereof, and a pharmaceutically acceptable excipient, wherein said pharmaceutical composition comprises from about 20 mg to about 200 mg of inorganic nitrite and is formulated as a solid dosage form for oral administration.

2. The method of claim 1, wherein administration of said pharmaceutical composition to said subject results in a plasma concentration of nitrite ion that is maintained between 0.1 μM and 5 μM for 4-14 hours.

3. The method of claim 1, wherein said method provides an improvement in pain, walking speed, walking distance, or stair climbing.

4. The method of claim 1, wherein said method provides an improvement in blood pressure.

5. The method of claim 1, wherein said subject is also diabetic.

6. The method of claim 5, wherein said method further provides an improvement in flow-mediated vasodilation.

7. The method of claim 1, wherein said inorganic nitrite is NaNO₂, KNO₂, or arginine nitrite.

8. The method of claim 7, wherein said inorganic nitrite is NaNO₂.

9. The method of claim 1, wherein said pharmaceutical composition is a tablet or capsule.

10. The method of claim 1, wherein said pharmaceutical composition comprises a pharmaceutically acceptable excipient for delayed release of the inorganic nitrite, or pharmaceutically acceptable salt thereof, such that, when orally administered to a subject, the inorganic nitrite or pharmaceutically acceptable salt thereof is not substantially released in the stomach of said subject.
